(19) United States
(12) Patent Application Publication
(10) Pub. No.: US 2018/0279606 A1
(43) Pub. Date: Oct. 4, 2018

(54) ANTIBACTERIAL AND ANTIFUNGAL ARTICLES, ANTIBACTERIAL AND ANTIFUNGAL AGRICULTURAL MATERIALS, AND ANTIBACTERIAL AND ANTIFUNGAL MEDICAL DEVICES

(71) Applicant: DAI NIPPON PRINTING CO., LTD., Tokyo-to (JP)

(72) Inventors: Yudai YAMASHITA, Tokyo-to (JP); Shigeki HATORI, Tokyo-to (JP); Masato TEZUKA, Tokyo-to (JP); Mihoko KURASHIGE, Tokyo-to (JP); Mikio ISHIKAWA, Tokyo-to (JP); Kaori YAMASHITA, Tokyo-to (JP)

(21) Appl. No.: 15/477,499

(22) Filed: Apr. 3, 2017

(51) Int. Cl. A01N 25/34; A61L 31/14; A61L 31/10

(52) U.S. Cl. CPC A01N 25/34; A61L 2300/404; A61L 31/10; A61L 31/14

(57) ABSTRACT

An antibacterial and antifungal article comprising a projection structure on a surface of the antibacterial and antifungal article, the projection structure comprising a projection group comprising a plurality of projections being disposed, where an average P_{AVG} of distances P between adjacent projections is 1 μm or less, wherein the projection structure comprises projections that a height H is 80 nm or more and 1000 nm or less and a ratio (Wt/Wb) of a width Wt at a 97% height of the height to a width Wb at a bottom, is 0.5 or less.

### TECHNICAL FIELD

The disclosure relates to antibacterial and antifungal articles, antibacterial and antifungal agricultural materials comprising the antibacterial and antifungal articles, and antibacterial and antifungal medical devices comprising the antibacterial and antifungal articles.

### BACKGROUND

To keep a clean environment, there is a need to provide antibacterial and antifungal properties (properties that can prevent the attachment of pathogens such as bacteria to the surface of articles and prevent the propagation of attached pathogens such as bacteria and fungi) to furniture, home electrical appliances, cooking appliances, medical equipment, articles such as food packaging materials, and interior materials for buildings, for example.

To provide antibacterial properties to various kinds of articles, for example, photocatalytic materials and antibacterial agents (e.g., silver ions) have been used. For example, Patent Literature 1 discloses water-repellent photocatalytic compositions and coating films thereof as materials aiming at providing both high stain resistance and high antimicrobial and antiviral properties even in weak light circumstances such as an indoor space, the water-repellent photocatalytic compositions comprising a water-repellent resin binder, a photocatalytic material and cuprous oxide, and the photocatalytic material being integrated with the cuprous oxide.

Patent Literature 2 discloses a composition as a material that can decompose and remove bacteria, viruses, germs or the like, the composition comprising a photocatalyst powder containing apatite with photocatalyst activity. Patent Literature 2 describes that when the surface of the photocatalyst powder is in a burr-like form, the surface area that serves as a photocatalyst increases and results in a further increase in contact efficiency with microorganisms.

Patent Literature 3 discloses such an antibacterial glass that the surface layer contains an antibacterial material and has a silver ion diffusion layer within a depth of 30 μm from the glass surface and a compression layer having a thickness of 15 μm or more in the depth direction from the glass surface.

Patent Literature 4 describes that by fixing an inorganic compound containing silver (1 μm or less in particle size) on fine concaves on the surface of a plastic film (having a surface roughness (Ra) of 0.2 μm or more, a maximum roughness (Rt) of 1 μm or more, and a surface roughness (Pc, i.e., the number of pieces having a height of 0.5 μm or more per mm) of 5 or more), the plastic film can prevent the detachment and removal of the inorganic compound, which is a compound with antibacterial properties, and can keep its antibacterial function over along period of time.

Patent Literature 5 describes an antibacterial decorative sheet comprising: a substrate sheet composed of a polyolefin-based resin; a design layer formed on the sheet; and a transparent or semi-transparent resin layer formed on the design layer, the transparent or semi-transparent resin layer containing an antibacterial agent. Patent Literature 5 also describes that a concavo-convex pattern can be formed on the resin layer by embossing.

In the agricultural field, in addition to traditional plastic greenhouse cultivation, there is a recent attempt to industrially produce agricultural products in doors by controlling temperature, humidity, light, etc., to a level that is stable for plant growth (plant factory cultivation). Plant factory cultivation is often carried out in a relatively closed space, and there is a small invasion of pathogens, fungi and the like. Accordingly, various attempts have been made to achieve pesticide-free production without the use of pesticides such as antibacterial agents and antifungal agents and with the use of a LED source, which has lower UV intensity than sunlight. However, once the invasion of bacteria or fungi is allowed by the entrance of people, etc., it may be difficult to eliminate the bacteria or fungi in the pesticide-free environment.
Patent Literature 1: Japanese Patent Application Laid-Open (JP-A) No. 2012-210557
Patent Literature 2: JP-A No. 2012-239499
Patent Literature 3: JP-A No. 2013-71878
Patent Literature 4: JP-A No. H09-57893
Patent Literature 5: JP-A No. H11-262983

### SUMMARY

Like the above-listed patent literatures 1 to 5, antibacterial agents have been used to provide antibacterial properties to various kinds of articles. Meanwhile, the inventors of the present invention studied other methods for providing antibacterial properties without the use of antibacterial agents, and they found that excellent antibacterial and antifungal properties can be provided by forming the surface of an article into a specific convexo-concave shape.

The disclosed embodiments were achieved based on the above knowledge. An object of the disclosed embodiments is to provide antibacterial and antifungal articles with excellent antibacterial and antifungal properties, agricultural materials comprising the antibacterial and antifungal articles, and medical devices comprising the antibacterial and antifungal articles.

In a first embodiment, there is provided an antibacterial and antifungal article comprising a projection structure on a surface of the antibacterial and antifungal article, the projection structure comprising a projection group comprising a plurality of projections being disposed, where an average P_{AVG} of distances P between adjacent projections is 1 μm or less, wherein the projection structure comprises projections that a height His 80 nm or more and 1000 nm or less and a ratio (Wt/Wb) of a width Wt at a 97% height of the height to a width Wb at a bottom, is 0.5 or less.

In a second embodiment, there is provided an antibacterial and antifungal article comprising a linear convexo-concave shape on a surface of the antibacterial and antifungal article, the linear convexo-concave shape comprising a plurality of linear convexes extending in one direction or approximately one direction, where an average P′_{AVG} of distances P′ between adjacent linear convexes is 1 μm or less, wherein the linear convexo-concave shape comprises linear convexes that a height H′ is 80 nm or more and 1000 nm or less and a ratio (Wt′/Wb′) of a width Wt′ at a 97% height of the height to a width Wb′ at a bottom, is 0.5 or less.

In other embodiments, there are provided antibacterial and antifungal agricultural materials and antibacterial and antifungal medical devices. At least a part of each antibacterial and antifungal agricultural material may comprise any one of the antibacterial and antifungal articles. At least a part of each antibacterial and antifungal medical device may comprise any one of the antibacterial and antifungal articles.

The disclosed embodiments can provide the antibacterial and antifungal articles with excellent antibacterial and antifungal properties, the agricultural materials comprising the antibacterial and antifungal articles, and the medical devices comprising the antibacterial and antifungal articles.

### BRIEF DESCRIPTION OF THE DRAWINGS

FIG. 1 is a schematic cross-sectional view of an example of the antibacterial and antifungal article according to an embodiment;

FIG. 2 is a schematic cross-sectional view of another example of the antibacterial and antifungal article according to an embodiment;

FIG. 3 is a schematic perspective view of another example of the antibacterial and antifungal article according to an embodiment;

FIG. 4 is an A-A′ cross-sectional view of the antibacterial and antifungal article shown in FIG. 3;

FIG. 5 is a schematic view of an example of a Delaunay diagram;

FIG. 6 is a photograph of a cross-section of an antibacterial and antifungal article of Example 1 taken by SEM;

FIG. 7 is a photograph of a cross-section of an antibacterial and antifungal article of Example 2 taken by SEM;

FIG. 8 is a photograph of a cross-section of an antibacterial and antifungal article of Example 3 taken by SEM;

FIG. 9 is a photograph of a cross-section of an antibacterial and antifungal article of Example 4 taken by SEM;

FIG. 10 is a photograph of a cross-section of an antibacterial and antifungal article of Example 5 taken by SEM;

FIG. 11 is a photograph of a cross-section of an antibacterial and antifungal article of Example 6 taken by SEM;

FIG. 12 is a photograph of a cross-section of a comparative article of Comparative Example 1 taken by SEM;

FIG. 13 is a photograph of a cross-section of a comparative article of Comparative Example 2 taken by SEM;

FIG. 14 is a photograph of a petri dish after antibacterial evaluation of the antibacterial and antifungal article of Example 1 using Escherichia coli;

FIG. 15 is a photograph of a petri dish after antibacterial evaluation of the antibacterial and antifungal article of Example 1 using Staphylococcus aureus;

FIG. 16 is a photograph of a petri dish after antibacterial evaluation of the comparative article of Comparative Example 1 using Escherichia coli;

FIG. 17 is a photograph of a petri dish after antibacterial evaluation of the comparative article of Comparative Example 1 using Staphylococcus aureus;

FIG. 18 is a schematic view of an example of the mode of use of the antibacterial and antifungal article according to an embodiment;

FIG. 19 is a schematic cross-sectional view of an example of a B-B′ cross-sectional view shown in FIG. 18;

FIG. 20 is a schematic view of another example of the mode of use of the antibacterial and antifungal article according to an embodiment;

FIG. 21 is a schematic cross-sectional view of an example of a D-D′ cross-sectional view shown in FIG. 20;

FIG. 22 is a schematic view of another example of the mode of use of the antibacterial and antifungal article according to an embodiment;

FIG. 23 is a schematic cross-sectional view of an example of an enlarged part of an F-F′ cross-section shown in FIG. 22;

FIG. 24 is a schematic perspective view of an example of the mode of use of the antibacterial and antifungal medical device according to an embodiment;

FIG. 25 is a schematic cross-sectional view of an example of a G-G′ cross-section shown in FIG. 24;

FIG. 26 is a schematic view of an example of the mode of use of the antibacterial and antifungal agricultural material according to an embodiment;

FIG. 27 is a schematic view of another example of the mode of use of the antibacterial and antifungal agricultural material according to an embodiment;

FIG. 28 is a schematic front view of another example of the mode of use of the antibacterial and antifungal medical device according to an embodiment;

FIG. 29 is a schematic cross-sectional view of an example of an H-H′ cross-section shown in FIG. 28;

FIG. 30 is a schematic cross-sectional view of another example of the H-H′ cross-section shown in FIG. 28;

FIG. 31 is an explanatory view of measurement fields in the measurement of a surface having a projection structure according to an embodiment; and

FIG. 32 is a schematic cross-sectional view of another example of the antibacterial and antifungal article according to an embodiment.

### DETAILED DESCRIPTION

Next, the embodiments of the disclosure will be described in detail. The disclosure is not limited to the following embodiments and may be carried out with arbitral modifications without deviating from the gist of the embodiments.

In this specification, “article” encompasses a variety of forms such as “plate”, “sheet” and “film”.

Also in this specification, terms used to specify shape, geometric condition and degrees thereof (such as “parallel”, “perpendicular” and “same”), terms relating to shape (such as “triangle” and “polygon”), values of length and angle, etc., are not interpreted in a strict sense and are interpreted in a sense that includes a certain amount of margin that can promise similar functions.

Also in this specification, (meth)acryl means each of acryl and methacryl; (meth)acrylate means each of acrylate and methacrylate; and (meth)acryloyl means each of acryloyl and methacryloyl.

Also in this specification, a cured product of a resin composition means a product solidified through or not through a chemical reaction.

The antibacterial and antifungal article according to the first embodiment is an antibacterial and antifungal article comprising a projection structure on a surface of the antibacterial and antifungal article, the projection structure comprising a projection group comprising a plurality of projections being disposed, where an average P_{AVG} of distances P between adjacent projections is 1 μm or less, wherein the projection structure comprises projections that a height H is 80 nm or more and 1000 nm or less and a ratio (Wt/Wb) of a width Wt at a 97% height of the height to a width Wb at a bottom, is 0.5 or less.

The antibacterial and antifungal article according to the second embodiment is an antibacterial and antifungal article comprising a linear convexo-concave shape on a surface of the antibacterial and antifungal article, the linear convexo-concave shape comprising a plurality of linear convexes extending in one direction or approximately one direction, where an average P′_{AVG} of distances P′ between adjacent linear convexes is 1 μm or less, wherein the linear convexo-concave shape comprises linear convexes that a height H′ is 80 nm or more and 1000 nm or less and a ratio (Wt′/Wb′) of a width Wt′ at a 97% height of the height to a width Wb′ at a bottom, is 0.5 or less.

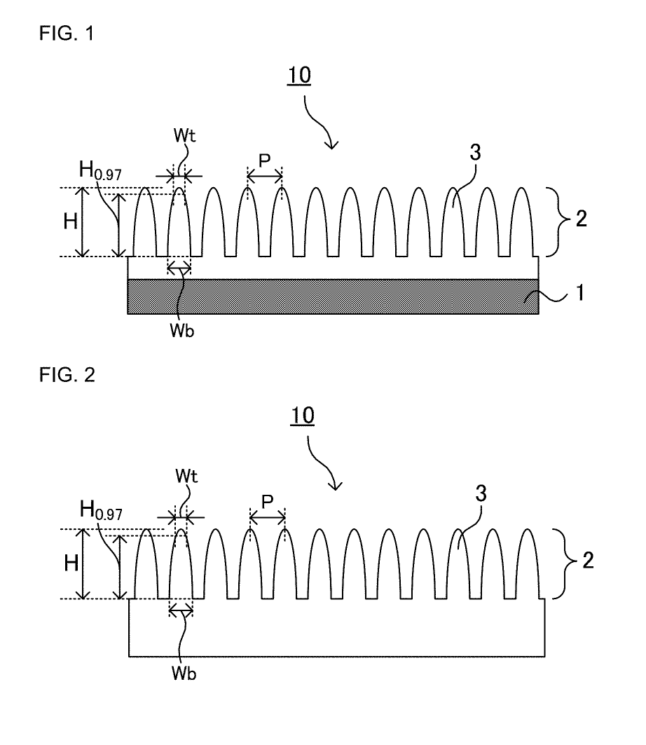

The antibacterial and antifungal articles according to the present disclosure will be described with reference to figures. FIG. 1 is a schematic cross-sectional view of an example of the antibacterial and antifungal article according to the first embodiment. An antibacterial and antifungal article 10 shown in FIG. 1 comprises, on a surface of a substrate 1, a projection structure 2 comprising a projection group that a plurality of projections are disposed. In the antibacterial and antifungal article 10 shown in FIG. 1, the projection structure 2 is formed into a convexo-concave layer composed of a different material from the substrate 1.

FIG. 2 is a schematic cross-sectional view of another example of the antibacterial and antifungal article according to the first embodiment. An antibacterial and antifungal article 10 shown in FIG. 2 comprises, on a surface thereof, a projection structure 2 comprising a projection group that a plurality of projections are disposed. The article does not have a substrate, or the projection structure 2 is integrated with a substrate.

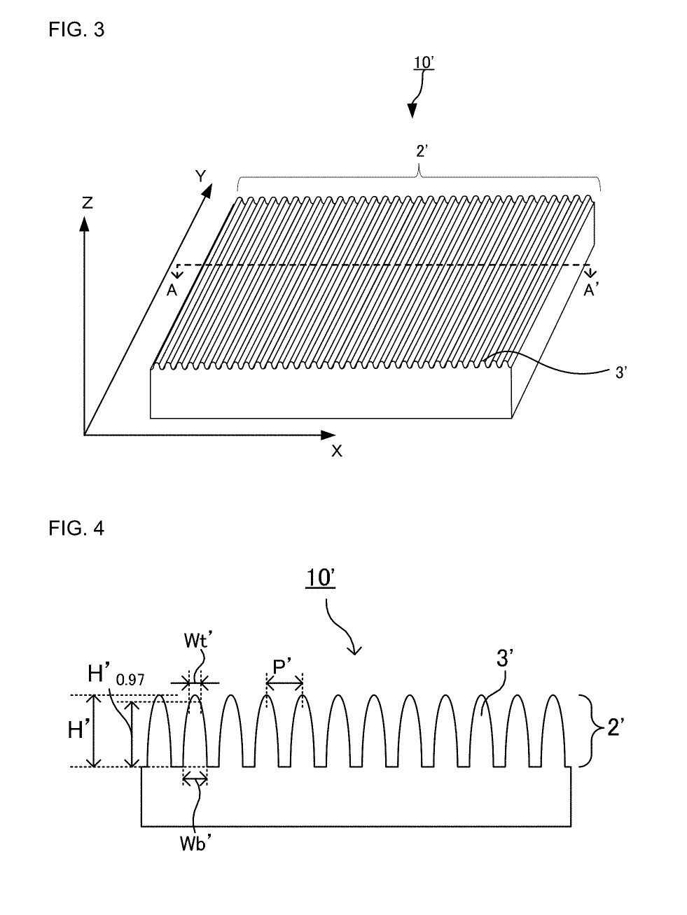

FIG. 3 is a schematic perspective view of another example of the antibacterial and antifungal article according to the second embodiment. FIG. 4 is an A-A′ cross-sectional view of the antibacterial and antifungal article shown in FIG. 3.

An antibacterial and antifungal article 10′ shown in FIGS. 3 and 4 comprises, on a surface thereof, a linear convexo-concave shape 2′ that a plurality of linear convexes 3′ extend in one direction or approximately one direction.

As shown in FIGS. 1 to 4, the antibacterial and antifungal articles according to the present disclosure may have the projection structure or linear convexo-concave shape on the whole surface. As shown in FIG. 32, the antibacterial and antifungal articles according to the disclosed embodiments may have the projection structure 2 or linear convexo-concave shape 2′ on at least a part of a surface of the substrate 1, or the projection structure 2 or linear convexo-concave shape 2′ may be disposed at intervals.

It is not still clear how the antibacterial and antifungal articles according to the present disclosure provide excellent antibacterial and antifungal properties; however, it is estimated as follows.

The antibacterial and antifungal article according to the first embodiment comprise, on a surface thereof, the projection structure comprising the projection group that the plurality of projections are disposed, and the average P_{AVG} of the distances P between the adjacent projections is 1 μm or less. Moreover, at least a part of the projections are such projections that the height H is 80 nm or more and 1000 nm or less, and the ratio (Wt/Wb) of the width Wt at the 97% height of the height (that is, H_{0.97}) to the width Wb at the bottom, is 0.5 or less.

The antibacterial and antifungal article according to the second embodiment comprises, on a surface thereof, the linear convexo-concave shape that the plurality of linear convexes extend in one direction or approximately one direction, and the average P′_{AVG} of the distances P′ between the adjacent linear convexes is 1 μm or less. Moreover, at least a part of the linear convexes are such linear convexes that the height H′ is 80 nm or more and 1000 nm or less, and the ratio (Wt′/Wb′) of the width Wt′ at the 97% height of the height to the width Wb′ at the bottom, is 0.5 or less.

The tips of both the projections and the linear convexes are in a tapered shape. In the antibacterial and antifungal article according to the first embodiment, the plurality of projections including the tapered-shaped projections are disposed at such intervals that the average P_{AVG} of the distances P between the adjacent projections is 1 μm or less. In the antibacterial and antifungal article according to the second embodiment, the plurality of linear convexes including the tapered-shaped linear convexes are disposed at such intervals that the average P′_{AVG} of the distances P′ between the adjacent linear convexes is 1 μm or less.

In general, bacteria are about 1 μm in size. Accordingly, when bacteria or fungi are attached to a surface having the projection structure or linear convexo-concave shape, the bacteria or fungi do not enter spaces between the projections or linear convexes, and they come into contact with the tips of the projections or linear convexes. As described above, the projections and linear convexes of the antibacterial and antifungal articles according to the present disclosure include the projections with the tapered-shaped tips and the linear convexes with the tapered-shaped tips, respectively. Therefore, it is considered that once bacteria or fungi attach to a surface having the projection structure or linear convexo-concave shape, the tips of the projections pierce the bacteria cells and kill the bacteria or fungi, thereby providing antibacterial and antifungal performance.

Hereinafter, the antibacterial and antifungal articles according to the present disclosure will be described in detail. The antibacterial and antifungal article according to the first embodiment and the antibacterial and antifungal article according to the second embodiment will be described in this order.

### <The Antibacterial and Antifungal Article According to the First Embodiment>

The antibacterial and antifungal article according to the first embodiment comprises the projection structure on a surface thereof. The antibacterial and antifungal article according to the present disclosure is typically a sheet-shaped antibacterial and antifungal article having the projection structure on the whole of one surface thereof. Also, it may be a sheet-shaped antibacterial and antifungal article having the projection structure on the whole of both surfaces thereof, or it may be a sheet-shaped antibacterial and antifungal article having the projection structure on a part of one surface thereof or on a part of each of both surfaces thereof. The antibacterial and antifungal article according to the present disclosure may have the projection structure on the whole surface thereof, in the case where the antibacterial and antifungal article is a molded product molded in a predetermined shape. For example, when the antibacterial and antifungal article is in a tube shape, it may have the projection structure on the inner surface of the tube. Also, the antibacterial and antifungal article according to the present disclosure may have the projection structure on a part of the surface. In this specification, “sheet-shaped” or “sheet shape” may be any of the following: one that can be bent and rolled up, one that cannot be bent and rolled up but can be curved by applying a load, and one that cannot be bent at all.

### (The Projection Structure)

The convexes of the projections constituting the projection structure are formed in an approximately vertical direction to a surface opposite to the surface at the side having the projection structure (hereinafter it may be simply referred to as back surface). In the case where the antibacterial and antifungal article according to the present disclosure is a molded product molded in the predetermined shape, the convexes are formed in an approximately vertical direction to the bottom surface of the projections.

For the projection structure according to the present disclosure, the average P_{AVG} of the distances P between the adjacent projections is 1 μm or less. The projection structure is a fine projection structure comprising such a fine projection group that the plurality of fine projections are disposed at the average P_{AVG} of the distances between the adjacent projections. The surface having the projection structure means that the surface has fine convexes and concaves. Since the P_{AVG} is 1 μm or less, bacteria or fungi effectively come into contact with the tips of the projections, and antibacterial and antifungal properties are provided. In the present disclosure, from the viewpoint of increasing antibacterial and antifungal properties, the average P_{AVG} of the distances P between the projections is preferably 500 nm or less, and more preferably 300 nm or less. From the viewpoint of obtaining the strength of the projections, the average P_{AVG} of the distances P between the projections is preferably 75 nm or more.

The adjacent projections relating to the distance P between the adjacent projections (hereinafter it may be referred to as “two adjacent projections' distance”) are so-called neighboring projections. Assuming that a region where the projections are distributed is partitioned into Voronoi regions using the plan-view-shaped centroid of each projection as a generating point, a projection belonging to a Voronoi region that is adjacent to the Voronoi region of another projection, is defined as a projection adjacent to another projection.

In the present disclosure, the average P_{AVG} of the two adjacent projections' distances P and the shape of the projections can be measured by an atomic force microscope (AFM), a scanning electron microscope (SEM) or a transmission electron microscope (TEM) and cross-section profile analysis.

The average P_{AVG} of the two adjacent projections' distances P is calculated by the following method.

(1) First, the in-plane arrangement of the projections (the plan-view shape of the projection arrangement) is detected using an atomic force microscope (AFM), a scanning electron microscope (SEM) or a transmission electron microscope (TEM).

(2) Then, the local maximum point of the height of each projection (hereinafter simply referred to as local maximum point) is detected from the thus-obtained in-plane arrangement. The local maximum point can be obtained by various methods such as a method of obtaining the local maximum point by sequentially comparing the plan-view shape to an enlarged photograph of a corresponding cross-sectional shape, and a method of obtaining the local maximum point by image processing of an enlarged plan-view photograph.

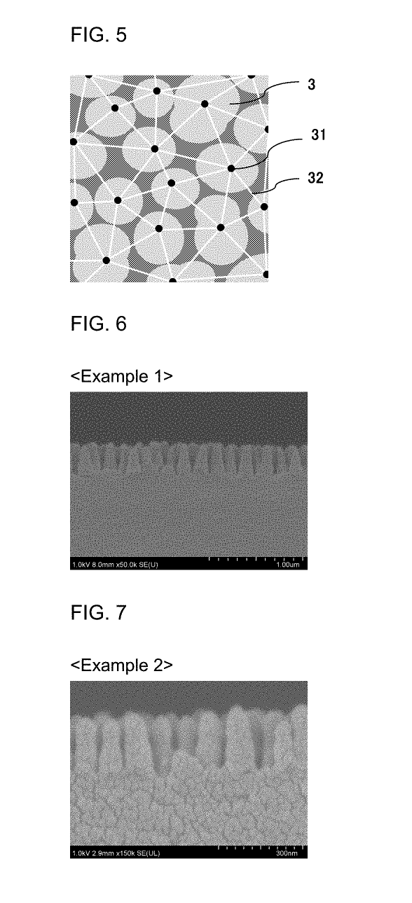

(3) Next, using the detected local maximum points as generating points, a Delaunary diagram is created. FIG. 5 shows a Delaunary diagram (a diagram represented by white line segments) overlapping with a schematic view of an enlarged plan view photograph of the antibacterial and antifungal article according to the present disclosure. As shown in FIG. 5, the Delaunay diagram is a network view that is composed of triangles that are obtained as follows: Voronoi partitioning was carried out using local maximum points 31 as generating points; generating points having adjacent Voronoi regions are defined as adjacent generating points; and the adjacent generating points are connected by line segments 32, thereby obtaining the triangles. The triangles are called Delaunay triangles, and the sides of the triangles (line segments connecting adjacent generating points) are called Delaunay lines.

(4) Next, the frequency distribution of the line segment lengths of Delaunay lines, that is, the frequency distribution of the distances between adjacent local maximum points (i.e., the frequency distribution of the two adjacent projections' distances) is obtained.

(5) The average value P_{AVG} can be obtained by regarding the thus-obtained frequency distribution of the two adjacent projections' distances P as a normal distribution.

In the present disclosure, at least a part of the projections are such projections that the height H is 80 nm or more and 1000 nm or less, and the ratio (Wt/Wb) of the width Wt at the 97% height of the height to the width Wb at the bottom, is 0.5 or less. In the present disclosure, since the projection structure comprises such projections, antibacterial and antifungal properties are provided.

From the viewpoint of excellent antibacterial and antifungal properties, such projections that the height H is 80 nm or more and 1000 nm or less, and the ratio (Wt/Wb) of the width Wt at the 97% height of the height to the width Wb at the bottom is 0.5 or less, are preferably 65% or more of all projections, more preferably 70% or more, even more preferably 85% or more, still more preferably 90% or more, yet more preferably 95% or more, and most preferably 98% or more. Also from the viewpoint of more excellent antibacterial and antifungal properties, it is particularly preferable that all (100%) of the projections constituting the projection structure are such projections that the height H is 80 nm or more and 1000 nm or less, and the ratio (Wt/Wb) of the width Wt at the 97% height of the height to the width Wb at the bottom, is 0.5 or less.

The height and width of each projection can be obtained by cross-section profile analysis. For each projection, the height H is determined as the distance in the vertical direction from the apex (that is, the highest point) to the bottom surface. The 97% height of the height (that is, H_{0.97}) means the height from the bottom surface to 97% when the height H is determined as 100% height.

The bottom surface of each projection is determined as a surface formed by connecting local minimum points at the base of the projection. The local minimum points at the base of each projection can be measured by use of a cross-section of the projection cut in the protruding direction of the projection.

The width of the projection at each height is determined as follows by cross-section profile analysis: horizontal cross-sections of the projection cut perpendicular to the height direction (that is, the vertical direction from the apex of the projection to the bottom surface of the same) at several heights, are created, and the maximum value of distances between two points on the profile of the cross-section for each height, is determined as the width of the projection at each height. For example, when the cross-section of the projection is elliptical, the width of the projection is the major axis of the ellipse.

Also in the present disclosure, the cross-section profile analysis can be carried out by use of a laser microscope, a three-dimensional optical profiler, etc. In particular, it can be carried out by use of LEXT OLS4100 (product name, manufactured by Olympus Corporation), ZeGage (product name, manufactured by Zygo) or the like.

Also in the present disclosure, in order to measure the surface having the projection structure, as shown in FIG. 31, a total of five fields (that is, fields a, b, c, d and e) are selected as the measurement fields from a whole surface A having the projection structure. The field a is a 1 mm-square field at the center. The field b, c, d and e are each a 1 mm-square field located at the midpoint of the center and an end point of a diagonal L1 or L2 of the surface A, the diagonal L1 passing through the center of the surface A, and the diagonal L2 being perpendicular to the diagonal L1.

Also in the present disclosure, when the surface having the projection structure of the antibacterial and antifungal article to be measured is larger than 1 meter square, the surface is cut to obtain a measurement sample that is 1 meter square in size, and the measurement sample is measured.

For the projections that the height H is 80 nm or more and 1000 nm or less, and the ratio (Wt/Wb) of the width Wt at the 97% height of the height to the width Wb at the bottom is 0.5 or less, the height H is preferably 100 nm or more and 500 nm or less, and more preferably 150 nm or more and 300 nm or less, from the viewpoint of antibacterial and antifungal properties and strength.

Also for the projections that the height H is 80 nm or more and 1000 nm or less, and the ratio (Wt/Wb) of the width Wt at the 97% height of the height to the width Wb at the bottom is 0.5 or less, the ratio Wt/Wb is preferably 0.4 or less, and more preferably 0.1 or more and 0.3 or less, from the viewpoint of antibacterial and antifungal properties.

The width Wb at the bottom and the width Wt at the 97% height of the height are widths shown in horizontal planes being perpendicular to the height direction. The width Wb at the bottom of each projection is the width of the bottom surface of the same.

From the viewpoint of antibacterial and antifungal properties, the ratio (H/Wb) of the height H of each projection to the width Wb at the bottom of the same, is preferably 0.4 or more, more preferably 0.8 or more, and still more preferably 1.0 or more. On the other hand, from the viewpoint of the strength of the projections, the ratio H/Wb is preferably 5.5 or less, more preferably 3.5 or less, still more preferably 2.5 or less, and particularly preferably 2.0 or less.

In the present disclosure, each projection preferably has the following structure: assuming that the projection is cut in horizontal planes being perpendicular to the height direction of the same, the cross-sectional area occupancy rate of a material part constituting the projection shown in the horizontal cross-sections, gradually increases from the apex of the projection to the bottom surface of the same, continuously along the height H of the projection. More preferably, each projection is in such a shape that the cross-sectional area occupancy rate absolutely converges to 0 at the apex.

As the shape of the projections, examples include, but are not limited to, those having vertical cross-sections in polygonal shapes (e.g., a triangle shape, a trapezoidal shape and a pentagonal shape), a pencil shape, a semicircular shape, a semi-elliptical shape, a parabolic shape, a bell shape, etc. From the viewpoint of excellent antibacterial and antifungal properties, the projections are preferably such that the vertical cross-section is in a polygonal, pencil or parabolic shape, more preferably such that the vertical cross-section is in a polygonal shape, and still more preferably such that the vertical cross-section is in a triangle shape. Such projections that the vertical cross-section is in a triangle shape, are typically in a circular cone shape or polyhedral cone shape. Such projections that the vertical cross-section is in a pencil shape, are typically in such a shape that a circular cone or polyhedral cone is placed on one end of a column or polygonal column so that the pointed top faces outward and the column or polygonal column is integrated with the circular cone or polyhedral cone. The projections may have the same shape or different shapes. As used herein, “vertical cross-section” means a cross-section that contains the apex of each projection and is parallel to the height direction of the projection.

For the antibacterial and antifungal article according to the present disclosure, no particular limitation is imposed on the number of the projections per unit area in a plan view of the surface having the projection structure. From the viewpoint of increasing antibacterial and antifungal properties, it is preferably 40000 per cm² or more, more preferably 100000 per cm² or more, and still more preferably 600000 per cm² or more. On the other hand, it is preferably 5000000 per cm² or less, more preferably 4000000 per cm² or less, and still more preferably 3000000 per cm² or less.

Also in the present disclosure, from the viewpoint of increasing antibacterial and antifungal properties, apart not comprising the above-specified projections is typically a substantially flat surface. However, the surface itself of the antibacterial and antifungal article may be curved or ridged. The substantially flat surface means that the surface may have such fine convexes and concaves that the height is 1/100 or less of the lower limit of the above-specified height H of the projections (e.g., fine convexes and concaves derived from scratches or raw materials).

For the antibacterial and antifungal article according to the present disclosure, a part of the surface may have convexes that are different from the above-specified projections, as long as the effect of the present disclosure is obtained.

For the antibacterial and antifungal article according to the present disclosure, the area on which the above-specified projections are disposed at the above-specified average two adjacent projections' distance P_{AVG}, is preferably 70% or more of the total area on which the projections are disposed, more preferably 80% or more, and still more preferably 90% or more.

Next, the material for the projection structure will be described. As the antibacterial and antifungal article according to the present disclosure, examples include, but are not limited to, (i) one comprising a substrate, a convexo-concave layer composed of a different material from the substrate, and the projection structure formed as the surface structure of the convexo-concave layer, (ii) one comprising a substrate and the projection structure composed of a different material from the substrate and formed on a surface of the substrate, (iii) one comprising a substrate and the projection structure composed of the same material as the substrate and integrated with the substrate to be formed on a surface of the substrate, and (iv) one comprising the projection structure formed on a surface of an article and not comprising a substrate. That is, in the present disclosure, the projection structure may be formed on a surface of a convexo-concave layer disposed on a support such as a substrate, may be integrated with a support such as a substrate, or may be directly formed on a surface of a substrate or article. The convexo-concave layer, substrate or article having the projection structure on a surface thereof, may have a monolayer or multilayer structure. The below-described material for the projection structure is a material for the projections constituting the projection structure, and it may be used in any of the convexo-concave layer, substrate and article having the projection structure on a surface thereof. The below-described material may be used to form only the projections; however, it is typically used to form the convexo-concave layer.

The material for the projection structure is not particularly limited, as long as it is a material that can form the projection structure. It can be appropriately selected depending on the intended application, and it may be a transparent or non-transparent material. As the material for the projection structure, examples include, but are not limited to, various kinds of resin compositions; rubbers such as fluorine rubber, butyl rubber, isoprene rubber, natural rubber and silicone rubber; glasses such as soda glass, potash glass, alkali-free glass and lead glass; ceramics such as lead lanthanum zirconate titanate (PLZT); inorganic materials such as quartz, fluorite and various kinds of metal oxides; metals such as silver, copper and iron, and alloys thereof; and combinations thereof.

The material for the projection structure is preferably a cured product of a resin composition, from the point of view that the shape of the projection group can be retained for a long period of time. The resin composition contains at least a resin and, as needed, other components such as a polymerization initiator. In the present disclosure, by using a cured product of a resin composition as the projection structure, and by appropriately controlling the composition of the resin composition, the shaping ability of the resin composition in the case of forming the projection structure by shaping, can be easily increased. Also, by adding various kinds of additives, antibacterial and antifungal properties can be easily increased further. Even in the case where various kinds of additives are added to the resin composition, by controlling the type and content of the resin or polymerization initiator, curing conditions for curing the resin composition (e.g., temperature and time) can be controlled to be within a range that does not alter the projection structure.

As the resin, examples include, but are not limited to, ionizing radiation curable resins such as (meth)acrylate-based, epoxy-based and polyester-based resins; thermosetting resins such as melamine-based, phenolic-based, polyester-based, (meth)acrylate-based, urethane-based, urea-based, epoxy-based and polysiloxane-based resins; and thermoplastic resins such as polyamide-based, polyolefin-based, polyvinyl chloride-based, (meth)acrylate-based, polyester-based, polycarbonate-based, polyethylene-based, polypropylene-based, polystyrene-based, polyurethane-based and nylon-based resins. Ionizing radiation means electromagnetic waves or charged particles that have an energy to polymerize and cure molecules. As the ionizing radiation, examples include, but are not limited to, all kinds of ultraviolet rays (UV-A, UV-B and UV-C), visible rays, gamma rays, X rays and electron beams. An ionizing radiation curable resin is obtained by appropriately intermixing a monomer, a polymer with a low degree of polymerization, or a reactive polymer, each of which contains a radically polymerizable and/or cationically polymerizable group per molecule. It is curable with a polymerization initiator.

From the viewpoint of providing excellent formability and mechanical strength to the projection structure, the resin composition is preferably an ionizing radiation curable resin composition containing an ionizing radiation curable resin, or a thermosetting resin composition containing a thermosetting resin. More preferably, an ionizing radiation curable resin composition.

Also, the resin composition preferably contains a (meth)acrylate-based resin. Since a (meth)acrylate-based resin can produce sterilizing gas, antibacterial properties can be increased.

Also, the resin composition is preferably a thermoplastic resin composition containing a thermoplastic resin, from the point of view that the resin composition can be molded by injection molding, extrusion molding or the like in this case. Also, the resin composition is preferably a thermoplastic resin composition containing a thermoplastic elastomer resin, from the point of view that a flexible molded product can be molded in this case.

### (The Ionizing Radiation Curable Resin Composition)

The ionizing radiation curable resin composition will be described in detail, using an ionizing radiation curable resin composition containing (meth)acrylate as an example, which is particularly preferably used among ionizing radiation curable resins that are suitable from the viewpoint of excellent formability and mechanical strength of the projections.

The (meth)acrylate may be a monofunctional (meth)acrylate having one (meth)acryloyl group per molecule, a polyfunctional (meth)acrylate having two or more (meth)acryloyl groups per molecule, or a combination thereof.

As the polyfunctional (meth)acrylate, examples include, but are not limited to, ethylene glycol di(meth)acrylate, diethylene glycol di(meth)acrylate, propylene di(meth)acrylate, hexanediol di(meth)acrylate, polyethylene glycol di(meth)acrylate, bisphenol A di(meth)acrylate, tetrabromo bisphenol A di(meth)acrylate, bisphenol S di(meth)acrylate, butanediol di(meth)acrylate, phthalic di(meth)acrylate, ethylene oxide-modified bisphenol A di(meth)acrylate, trimethylolpropane tri(meth)acrylate, pentaerythritol tri(meth)acrylate, pentaerythritol tetra(meth)acrylate, tris(acryloxyethyl)isocyanurate, dipentaerythritol hexa(meth)acrylate, urethane tri(meth)acrylate, ester tri(meth)acrylate, urethane hexa(meth)acrylate, and ethylene oxide-modified trimethylolpropane tri(meth)acrylate.

The content of the polyfunctional (meth)acrylate is preferably 40% by mass or more and 99.9% by mass or less of the total solid content of the ionizing radiation curable resin composition, and more preferably 50% by mass or more and 99.5% by mass or less. In the case where the polyfunctional (meth)acrylate is used in combination with the below-described monofunctional (meth)acrylate, the content is 40% by mass or more and 98.9% by mass or less of the total solid content of the ionizing radiation curable resin composition, and more preferably 50% by mass or more and 96.5% by mass or less. In this specification, “solid content” means components other than solvents.

As the mono functional (meth)acrylate, examples include, but are not limited to, methyl (meth)acrylate, hexyl (meth)acrylate, decyl (meth)acrylate, allyl (meth)acrylate, benzyl (meth)acrylate, butoxyethyl (meth)acrylate, butoxyethylene glycol (meth)acrylate, cyclohexyl (meth)acrylate, dicyclopentanyl (meth)acrylate, 2-ethylhexyl (meth)acrylate, glycerol (meth)acrylate, glycidyl (meth)acrylate, 2-hydroxyethyl (meth)acrylate, 2-hydroxypropyl (meth)acrylate, isobornyl (meth)acrylate, isodecyl (meth)acrylate, isooctyl (meth)acrylate, lauryl (meth)acrylate, 2-methoxyethyl (meth)acrylate, methoxyethylene glycol (meth)acrylate, phenoxyethyl (meth)acrylate, stearyl (meth)acrylate, dodecyl (meth)acrylate, tridecyl (meth)acrylate, biphenyloxy ethyl acrylate, bisphenol A diglycidyl (meth)acrylate, biphenyloxy ethyl (meth)acrylate, ethylene oxide-modified biphenyloxy ethyl (meth)acrylate, and bisphenol A epoxy (meth)acrylate. These monofunctional (meth)acrylic esters may be used alone or in combination of two or more kinds.

In the case of using the monofunctional (meth)acrylate, the content of the monofunctional (meth)acrylate is preferably 1% by mass or more and 30% by mass or less of the total solid content of the ionizing radiation curable resin composition, and more preferably 3% by mass or more and 15% by mass or less.

To initiate or promote a curing reaction of the (meth)acrylate, as needed, a photopolymerization initiator may be appropriately selected and used. In the case of a radically polymerizable, ionizing radiation curable resin such as a (meth)acrylate-based resin, as the photopolymerization initiator, examples include, but are not limited to, bisacyl phosphine oxide, 1-hydroxycyclohexyl phenyl ketone, 2-hydroxy-2-methyl-1-phenylpropane-1-one, 2,2-dimethoxy-1,2-diphenylethane-1-one, 1-[4-(2-hydroxyethoxy)-phenyl]-2-hydroxy-2-methyl-1-propane-1-one, 2-methyl-1-[4-(methylthio)phenyl]-2-morpholinopropane-1-one, 2-benzyl-2-dimethylamino-1-(4-morpholinophenyl)-butanone-1, 2-hydroxy-2-methyl-1-phenyl-propane-1-ketone, 2,4,6-trimethylbenzoyl diphenyl phosphine oxide, phenylbis(2,4,6-trimethylbenzoyl)-phosphine oxide, and phenyl(2,4,6-trimethylbenzoyl)phosphinic acid ethyl ester. In the case of a cationically polymerizable ionizing radiation curable resin such as an epoxy-based resin, as the photopolymerization initiator, examples include, but are not limited to, aromatic iodonium salts and metallocene-based compounds. They may be used alone or in combination of two or more kinds.

In the case of using the photopolymerization initiator, generally, the content of the photopolymerization initiator is preferably 0.1% by mass or more and 10% by mass or less of the total solid content of the ionizing radiation curable resin composition, and more preferably 0.5% by mass or more and 5% by mass or less.

The ionizing radiation curable resin composition may further contain other components, to the extent that does not impair the effect of the present disclosure. As the other components, examples include, but are not limited to, a surfactant for wettability control, a fluorine-based compound, a silicone-based compound, a stabilizer, a defoaming agent, a cissing inhibitor, an antioxidant, an aggregation inhibitor, a viscosity modifier and a release agent.

A member comprising the projection group on a surface thereof, may be surface-treated. For example, to control wettability, a vapor-deposited film of a fluorine-based compound, a silicone-based compound or the like may be formed on the surface having the projection group.

### (The Thermoplastic Resin Composition)

The thermoplastic resin may be appropriately selected depending on the intended application and shape of the antibacterial and antifungal article, and it is not particularly limited. As the thermoplastic resin, examples include, but are not limited to, polyurethane, polystyrene, polyvinyl chloride, acrylonitrile-butadiene-styrene (ABS) resin, polymethylpentene, polycarbonate, polyimide, nylon, polysulfone, polypropylene, fluorine resin, polyethylene, polyether ketone, polyphenyl sulfone, polyarylamide, polyaryl ether ketone, liquid crystal polymer, ionomer resin, self-reinforced polyphenylene (SRP) and thermoplastic elastomer. As the thermoplastic elastomer, examples include, but are not limited to, polyolefin-based, nylon-based, polystyrene-based and polyester-based elastomers. Of these examples, nylon-based, polyurethane-based, polyester-based, and polyolefin-based thermoplastic resins and elastomers are preferred.

The thermoplastic resin composition containing the thermoplastic resin may contain other components. However, from the viewpoint of preventing the elution of impurities, the content of the thermoplastic resin is preferably 99.0% by mass or more, and more preferably 99.5% by mass or more.

As the material for the projection structure, commercially-available products may be used. In the case where the antibacterial and antifungal article according to the present disclosure is used for applications where a low elution of impurities is needed during use (e.g., medical devices, cell culture vessels, experimental devices, food or beverage containers or packages, and cooking devices), a material with a low content of impurities is preferably used. As the material with a low content of impurities, a material with lower values than the standard values defined by the combustion tests and the test for extractable substances of “Test Methods for Plastic Containers” in the Japanese Pharmacopoeia (14th Edition) is used. More specifically, a material satisfying the following conditions is used:

(Combustion Tests)
Residue on ignition: Not more than 0.1%
Heavy metals: Not more than detection limit (20 μg/g)

(Test for Extractable Substances)
Time required for foam to disappear: Within 180 seconds
ΔpH: Not more than 1.5
Potassium permanganate-reducing substances: Not more than 1.0 mL
UV spectrum: The maximum absorbance between 220 nm and 240 nm is not more than 0.08, and the maximum absorbance between 241 nm and 350 nm is not more than 0.05.
Residue on evaporation: Not more than 1.0 mg

As the material with a low content of impurities, examples include, but are not limited to, polyvinyl chloride (PVC), low density polyethylene (LDPE), high density polyethylene (HDPE), polypropylene (PP), acrylonitrile/butadiene/styrene copolymer resin (ABS), polycarbonate (PC) and polyethylene terephthalate (PET). In addition, various kinds of thermoplastic elastomers (TPE), polystyrene (PS), cycloolefin polymer (COP) resin, and special plastics such as polysulfone and silicone may be used in some cases. As the commercially-available products, examples include, but are not limited to, thermoplastic resins such as Somos and Evolve (product names, manufactured by DSM), EPO-TEK (product name, available from Rikei Corporation), 211-CTH-SC (product name, manufactured by DYMAX), TPX (product name, manufactured by Mitsui Chemicals, Inc.), Udel, KetaSpire, Radel, lxef, AvaSpire and PrimoSpire (product names, manufactured by SOLVAY), Dyneema Purity (product name, manufactured by DSM), NEWCON and NOVATEC-PP (product names, manufactured by Japan Polypropylene Corporation), lupilon and NOVAREX (product names, manufactured by Mitsubishi Engineering-Plastics Corporation), HIMILAN (product name, manufactured by DuPont-Mitsui Polychemicals Co., Ltd.), SURLYN (product name, manufactured by DuPont), Hytrel (product name, manufactured by DuPont-Toray Co., Ltd.), ZELAS and RABALON (product names, manufactured by Mitsubishi Chemical Corporation) and ZEONOR (product name, manufactured by ZEON Corporation).

### (The Substrate)

The antibacterial and antifungal article according to the present disclosure may comprise the substrate as a support. The substrate used in the present disclosure can be appropriately selected depending on the intended application, and it may be a transparent or non-transparent substrate and is not particularly limited. As the material for the transparent substrate, examples include, but are not limited to, acetyl cellulose-based resins such as triacetyl cellulose; polyester-based resins such as polyethylene terephthalate and polyethylene naphthalate; olefin-based resins such as polyethylene and polymethylpentene; (meth)acrylic-based resins; polyurethane-based resins; resins such as polyethersulfone, polycarbonate, polysulfone, polyether, polyether ketone, acrylonitrile, methacrylonitrile, cycloolefin polymer and cycloolefin copolymer; glasses such as soda glass, potash glass, alkali-free glass and lead glass; ceramics such as lead lanthanum zirconate titanate (PLZT); and transparent inorganic materials such as quarts and fluorite. As the material for the non-transparent substrate, examples include, but are not limited to, metal, paper, fabric, wood, stone and composite materials thereof, and composite materials of them with the materials for the transparent substrate.

In the case where the substrate and the projection structure are integrated with each other, as the material for the substrate, examples include, but are not limited to, thermoplastic resins and resin compositions used as the materials for the above-described projection structure.

The substrate may be a sheet or film. Also, it may be any one of the following: one that can be rolled up, one that cannot be bent and rolled up but can be curved by applying a load, and one that cannot be bent at all. The thickness of the substrate can be appropriately selected depending on the intended application, and it is not particularly limited. In general, the thickness is from 10 μm or more and 5000 μm or less.

The structure of the substrate used for the antibacterial and antifungal article according to the present disclosure, is not limited to a monolayer structure and may be a multilayer structure. When the substrate has a multilayer structure, the multilayer structure may be composed of layers of the same composition or layers of different compositions.

When the projection structure is formed into a convexo-concave layer composed of a different material from the substrate, a primer layer may be formed on the substrate to increase adhesion between the substrate and the convexo-concave layer and increase abrasion resistance (scratch resistance). When the substrate is a transparent substrate, the primer layer is preferably one having visible light permeability and adhesion to the convexo-concave layer that is adjacent to the transparent substrate via the primer layer. When interference fringes are produced due to a refractive index difference between the transparent substrate and the convexo-concave layer, the interference fringes can be reduced by controlling the refractive index of the primer layer to a value intermediate between the refractive index of the substrate and that of the convexo-concave layer.

For the substrate used for the antibacterial and antifungal article according to the present disclosure, the total light transmittance in the visible range can be appropriately controlled depending on the intended application, and it is not particularly limited. For example, a transparent substrate having a total light transmittance of 80% or more may be used, a semi-transparent substrate having a total light transmittance of less than 80%, or a non-transparent substrate may be used. The total light transmittance can be measured in accordance with JIS K7361-1 (“Plastics: Determination of the total light transmittance of transparent materials”).

For example, when the antibacterial and antifungal article according to the present disclosure is used as a transparent member such as a protection film, the substrate is preferably a transparent substrate. Even when the antibacterial and antifungal article according to the present disclosure is used in such a manner that the article is attached to something before use, the substrate is preferably a transparent substrate, in order not to hinder a design.

When the antibacterial and antifungal article according to the present disclosure is placed on a glass part, the substrate is preferably a polyester-based resin substrate such as polyethylene terephthalate (PET) from the viewpoint of providing shatter resistance in the case of breakage of the glass part.

The antibacterial and antifungal article according to the present disclosure may be a laminate of the antibacterial and antifungal article and an adhesive layer. The adhesive layer is typically disposed at a side not having the projection structure of the antibacterial and antifungal article. When the antibacterial and antifungal article according to the present disclosure comprises the adhesive layer, to attach the antibacterial and antifungal article according to the present disclosure to different articles, etc., the adhesive layer may be disposed on the outermost surface or under a removable protection film that will be described below. When the antibacterial and antifungal article according to the present disclosure has a multilayer structure composed of two or more layers, the adhesive layer may be disposed between the layers to attach them.

The material for the adhesive layer may be a known adhesive and is not particularly limited.

The antibacterial and antifungal article according to the present disclosure may have the removable protection film on at least a part of the surface. The antibacterial and antifungal article according to the present disclosure may be in such a form that, having the removable protection film temporarily attached to at least a part of the surface, the antibacterial and antifungal article is stored, transported, traded, and post-processed or installed, and the protection film is removed therefrom at an appropriate time.

The antibacterial and antifungal article according to the present disclosure is not particularly limited. Depending on the intended application, the total light transmittance in the visible range can be 80% or more. Since the total light transmittance is equal to or more than the lower limit, in the case of attaching the antibacterial and antifungal article according to the present disclosure to a different article for use, damage to the design of the base surface is prevented, and excellent visibility is obtained. The total light transmittance can be measured in accordance with JIS K7361-1 (“Plastics: Determination of the total light transmittance of transparent materials”).

The static contact angle of water with the surface of the antibacterial and antifungal article according to the first embodiment, is not particularly limited. The antibacterial and antifungal article can provide excellent antibacterial and antifungal properties even when the contact angle of water with the surface having the projection structure is more than 10 degrees and less than 120 degrees, according to the θ/2 method. In general, when the contact angle of water is more than 10 degrees and less than 120 degrees according to the θ/2 method, water easily remains on the surface and tends to deteriorate the antibacterial and antifungal properties of the antibacterial and antifungal article. For the antibacterial and antifungal article according to the present disclosure, the angle of water with the surface having the projection structure is preferably 40 degrees or more and 100 degrees or less, more preferably 45 degrees or more and 85 degrees or less, and still more preferably 60 degrees or more and 80 degrees or less, according to the θ/2 method, from the point of view that both projection strength and antibacterial and antifungal properties are easily provided.

In the present disclosure, the static contact angle of water is a contact angle measured according to the θ/2 method in which 1.0 μL of pure water is dropped on a surface of a measuring object, and one second after the water droplet reaches the surface, the contact angle is calculated from angles formed by the (solid) surface and the straight line connecting the top of the droplet to the right or left edge point of the same. As the measurement device, for example, contact angle meter DM 500 (product name, manufactured by Kyowa Interface Science Co., Ltd.) may be used.

### (The Method for Producing the Antibacterial and Antifungal Article According to the First Embodiment)

The method for producing the antibacterial and antifungal article according to the present disclosure may be a method that can produce the above-described antibacterial and antifungal article according to the present disclosure. It may be appropriately selected depending on the material for the antibacterial and antifungal article, the intended application of the same, etc., and it is not particularly limited. As the method, examples include, but are not limited to, a shaping method, a blasting method, a photolithography method, a tool cutting method, combinations thereof, an injection molding method, a calendering method and an extrusion molding method. From the viewpoint of formability, in the case of forming the projection structure using the ionizing radiation-curable resin composition, a method for shaping the convexo-concave shape of an original plate for forming the projection structure, is preferred. In the case of forming the projection structure using the thermoplastic resin composition, an injection or extrusion molding method using the original plate for forming the projection structure as a mold, is preferred.

As the method for producing the antibacterial and antifungal article according to the present disclosure by shaping the convexo-concave shape of the original plate for forming the projection structure, examples include, but are not limited to, the following method: an original plate for forming the projection structure is prepared, which has a convexo-concave-shaped surface having many pores formed thereon (the convexo-concave shape of the convexo-concave-shaped surface corresponds to the shape of the surface having the projection structure of the antibacterial and antifungal article according to the present disclosure); the convexo-concave-shaped surface of the original plate for forming the projection structure is pressed to a surface of a coating film of the resin composition for forming the projection structure; and the coating film of the resin composition is cured and then removed from the original plate for forming the projection structure, thereby forming the desired projection structure by shaping. The method for curing the resin composition can be appropriately selected depending on the type and so on of the resin composition. In the case of using the thermoplastic resin composition containing the thermoplastic resin as the resin composition for forming the projection structure, the resin composition is heated at a temperature appropriately selected depending on the softening temperature of the thermoplastic resin; the convexo-concave-shaped surface of the original plate for forming the projection structure, is pressed to a surface of the thermoplastic resin composition to shape the projection structure; and the resin composition is solidified by cooling and then removed from the original plate for forming the projection structure, thereby forming the desired projection structure on the surface of the thermoplastic resin composition by shaping.

The original plate for forming the projection structure is not particularly limited, as long as it is resistant to deformation and abrasion even after repeated use. It may be a metal or resin plate. In general, it is preferably a metal plate, since a metal plate has excellent resistance to deformation and abrasion.

As the method for forming the convexo-concave shape on the original plate for forming the projection structure, examples include, but are not limited to, an anodization method, a photolithography method, a laser lithography method, an electron beam lithography method, a blasting method and combinations thereof.

In the case of forming the convexo-concave shape on the original plate for forming the projection structure by the anodization method, the convexo-concave-shaped surface of the original plate for forming the projection structure is preferably composed of aluminum, from the point of view that it can be processed easily by anodization. As the original plate, examples include, but are not limited to, an original plate obtained by providing a high-purity aluminum layer on a surface of a parent material composed of a metal (e.g., stainless-steel, copper, aluminum) directly or via any of various kinds of intermediate layers by sputtering, etc. The convexo-concave shape may be formed on the aluminum layer. Before providing the aluminum layer, the surface of the parent material may be highly mirror polished by a composite electrolytic polishing method using a combination of an electrodissolution effect and an abrasive friction effect.

As the method for forming the convexo-concave shape on the original plate for forming the projection structure by the anodization method, examples include, but are not limited to, a method of sequentially repeating an anodization step (a step of forming fine pores on a surface of the aluminum layer by the anodization method), a first etching step (a step of providing a tapered shape to the openings of the fine pores by etching the aluminum surface) and a second etching step (a step of increasing the pore diameter of the fine pores by etching the aluminum layer at a higher etching rate than that of the first etching step).

In the case of forming the convexo-concave shape on the original plate for forming the projection structure by the anodization method, the desired convexo-concave shape can be formed by appropriately control the purity (impurity amount) of the aluminum layer, the crystal particle diameter of the same, and the anodization and/or etching conditions. More specifically, by controlling liquid temperature, applied voltage, anodization time, etc., in the anodization step, a desired depth and a desired shape can be provided to the fine pores.

In the case of forming the convexo-concave shape by the anodization method, many fine pores are densely formed on the original plate for forming the projection structure. The projection structure produced using the original plate for forming the projection structure, comprises a projection group in which projections having a shape corresponding to the fine pores are densely disposed.

In the case of forming the convexo-concave shape on the original plate for forming the projection structure by any one of a photolithography method, a laser lithography method, an electron beam lithography method and combinations thereof, the parent material for the original plate for forming the projection structure is preferably a silicon wafer or a parent material composed of stainless-steel, aluminum or the like and uniformly plated with chromium or copper. More specifically, a resist layer is formed by spin-coating an appropriately selected resin resist on the parent material, or in the case of using a silicon wafer as the parent material, a surface of the silicon wafer is thermally oxidized to form a silicon oxide film that serves as a mask for silicon etching. Then, a resist pattern is formed by any one of a photolithography method, a laser lithography method, an electron beam lithography method and combinations thereof. In the case of using the resin resist, excess of the resin resist is removed by a developing treatment using a predetermined developer. Then, dry etching is applied to the metal film or silicon oxide film exposed at the openings of the thus-formed resist pattern. As needed, using a resist pattern layer and a metal pattern layer as etching-resistant layers, dry etching is applied to the parent material, followed by removal of the resist. In the case of using the silicon wafer as the parent material, inverted pyramid-shaped pores can be formed by crystal anisotropic etching. Therefore, the original plate for forming the projection structure having the desired convexo-concave shape formed thereon, can be obtained.

In the case of forming the convexo-concave shape on the original plate for forming the projection structure by blasting, as the parent material for the original plate for forming the projection structure, examples include, but are not limited to, metal plates such as a stainless-steel plate and an aluminum plate. A plating film such as a chromium plating film or a copper plating film may be formed on the blasted surface of the parent material.

In addition, the original plate for forming the projection structure may be uniformly coated with a thin film such as a diamond-like carbon (DLC) thin film, in order to increase the durability of the original plate.

The shape of the original plate for forming the projection structure used for shaping, is not particularly limited, as long as it can shape the desired convexo-concave shape. For example, the original plate may be a flat or rolled plate. In the present disclosure, from the point of view that the projection structure can be easily formed, a flat plate-shaped mold is preferably used as the original plate for forming the projection structure used for shaping. By the use of the flat plate-shaped mold, deformation of the projections or deformation of the projection structure due to adherence of the projections to each other, can be easily prevented when the mold is removed from the cured product of the resin composition.

As the flat plate-shaped mold used in the present disclosure, examples include, but are not limited to, such a mold that, using a plate-shaped metal material as the parent material, a convexo-concave shape corresponding to the shape of the projection structure is formed on an aluminum layer by, as described above, repeating the anodization treatment and the etching treatment, the aluminum layer being provided on the surface of the parent material directly or via any of various kinds of intermediate layers.

In the case of producing the antibacterial and antifungal article according to the present disclosure by the injection or extrusion molding method using the thermoplastic resin composition, for example, the original plate for forming the projection structure produced by the above-described method, can be formed into the shape of the mold of an injection or extrusion molding machine by a desired method and then used. To increase releasability, a release treatment is preferably carried out on the surface of the mold for the injection or extrusion molding. The release treatment may be carried out by a known method and is not particularly limited. As the release treatment, examples include, but are not limited to, applying of a release agent to the surface and coating of the surface with a thin DLC film.

As the method for producing the antibacterial and antifungal article according to the present disclosure by the injection molding method, examples include, but are not limited to, the following method: a mold having a core and a cavity and having a convexo-concave shape corresponding to the projection structure on the surface of at least one of the core and the cavity, is used as a mold for the injection molding; a hollow formed by the core and the cavity is filled with a thermoplastic resin composition melted by heating; the resin composition is solidified by cooling; the mold for the injection molding is released therefrom, thereby obtaining a molded product. In the case of producing a tube-shaped molded product having the projection structure on the inner surface thereof as the antibacterial and antifungal article according to the present disclosure, as the method for producing the tube-shaped molded product, examples include, but are not limited to, the above-mentioned method in which, however, such a mold for injection molding is used, that the hollow formed by the core and the cavity is in a tube shape, and the core surface has a convexo-concave shape corresponding to the projection structure thereon.

As the method for producing the tube-shaped molded product having the projection structure on the inner surface thereof by the extrusion molding method, examples include, but are not limited to, the following method: using a cored bar which has a convexo-concave shape corresponding to the projection structure on the surface thereof, according to a known wire coating method, the surface of the cored bar is coated with the thermoplastic resin composition by extrusion molding, followed by pulling out of the cored bar. As the method for coating the cored bar with the thermoplastic resin composition, examples include, but are not limited to, extrusion molding, coating and dipping. As the method for pulling out the cored bar, examples include, but are not limited to, the following method: the cored bar is extended to decrease the diameter; the molded product is removed from the cored bar; and then the cored bar is pulled out.

### <The Antibacterial and Antifungal Article According to the Second Embodiment>

The antibacterial and antifungal article according to the second embodiment comprises the linear convexo-concave shape on a surface thereof. The antibacterial and antifungal article according to the present disclosure is typically a sheet-shaped antibacterial and antifungal article having the linear convexo-concave shape on the whole of one surface thereof. Also, it may be a sheet-shaped antibacterial and antifungal article having the linear convexo-concave shape on the whole of both surfaces thereof, or it may be a sheet-shaped antibacterial and antifungal article having the linear convexo-concave shape on a part of one surface thereof or on apart of each of both surfaces thereof. The antibacterial and antifungal article according to the present disclosure may have the linear convexo-concave shape on the whole surface thereof, in the case where the antibacterial and antifungal article is a molded product molded in a predetermined shape. For example, when the antibacterial and antifungal article is in a tube shape, it may have the linear convexo-concave shape on the inner surface of the tube. Also, the antibacterial and antifungal article according to the present disclosure may have the linear convexo-concave shape on a part of the surface.

The linear convexes constituting the linear convexo-concave shape are formed in an approximately perpendicular direction with respect to a surface opposite to the surface having the linear convexo-concave shape (hereinafter it may be simply referred to as back surface) or, when the antibacterial and antifungal article according to the present disclosure is a molded product molded in a predetermined shape, the linear convexes are formed in an approximately vertical direction with respect to the bottom surface of the linear convexo-concave shape.

For the linear convexo-concave shape according to the present disclosure, the average P′_{AVG} of distances P′ between adjacent linear convexes is 1 μm or less (hereinafter, the distance between the adjacent linear convexes may be referred to as “two adjacent linear convexes' distance”). The linear convexo-concave shape is a linear fine convexo-concave shape comprising such a linear convex group that the plurality of linear convexes are disposed at the average P′_{AVG} of the two adjacent linear convexes' distances. A surface having the linear convexo-concave shape means that the surface has fine convexes and concaves. Since the P′_{AVG} is 1 μm or less, bacteria or fungi efficiently come into contact with the tips of the linear convexes. Therefore, antibacterial properties are provided. In the present disclosure, from the viewpoint of increasing antibacterial and antifungal properties, the average P′_{AVG} of the distances P′ between the linear convexes is preferably 500 nm or less, and more preferably 300 nm or less. From the viewpoint of obtaining the strength of the linear convexes, the average P′_{AVG} of the distances P′ between the linear convexes is preferably 100 nm or more.

The distance P′ between the adjacent linear convexes is determined as the distance between the apices of the linear convexes in a direction perpendicular to the extending direction of the linear convexes.

In the present disclosure, at least a part of the linear convexes are such linear convexes that the height H′ is 80 nm or more and 1000 nm or less, and the ratio (Wt′/Wb′) of the width Wt′ at the 97% height of the height to the width Wb′ at the bottom, is 0.5 or less. In the present disclosure, since the linear convexo-concave shape includes such linear convexes, antibacterial and antifungal properties are provided.

From the viewpoint of excellent antibacterial and antifungal properties, such linear convexes that the height H′ is 80 nm or more and 1000 nm or less, and the ratio (Wt′/Wb′) of the width Wt′ at the 97% height of the height to the width Wb′ at the bottom, is 0.5 or less, are preferably 95% or more of all linear convexes, and more preferably 98% or more. Also from the viewpoint of excellent antibacterial and antifungal properties, it is particularly preferable that all (100%) of the linear convexes constituting the linear convexo-concave shape are such linear convexes that the height H′ is 80 nm or more and 1000 nm or less, and the ratio (Wt′/Wb′) of the width Wt′ at the 97% height of the height to the width Wb′ at the bottom, is 0.5 or less.

In the present disclosure, the average P′_{AVG} of the two adjacent linear convexes' distances P′ and the shape of the linear convexes (e.g., height and width) can be measured by cross-section profile analysis using an atomic force microscope (AFM), a scanning electron microscope (SEM) or a transmission electron microscope (TEM). For each linear convex, the height H′ is determined as the distance in the vertical direction from the apex (that is, the highest point) to the bottom. The 97% height of the height (that is, H′_{0.97}) means the height from the bottom to 97% when the height H′ of each linear convex is determined as 100% height.

The bottom of each linear convex is determined as the position of a line segment formed by connecting local minimum points at the base of the linear convex shown on a cross-section of the linear convex cut in a direction perpendicular to the extending direction of the linear convex.

The width of the linear convex at each height is determined as the distance between two points on a profile at each height shown on a cross-section of the linear convex cut in a direction perpendicular to the extending direction of the linear convex.

In the second embodiment, the cross-section profile analysis can be carried out by use of the same analysis as the first embodiment. In the measurement of the surface having the linear convexo-concave shape, the measurement fields can be the same as the measurement of the surface having the projection structure according to the first embodiment.

For the linear convexes that the height H′ is 80 nm or more and 1000 nm or less, and the ratio (Wt′/Wb′) of the width Wt′ at the 97% height of the height to the width Wb′ at the bottom is 0.5 or less, the height H′ is preferably 100 nm or more, from the viewpoint of antibacterial and antifungal properties. From the viewpoint of strength, the height H′ is preferably 900 nm or less, more preferably 500 nm or less, and still more preferably 300 nm or less. When the width Wb′ at the bottom is 300 nm or more, the antibacterial and antifungal article according to the second embodiment has particularly excellent antifungal properties and can be preferably used as an antifungal article. In this case, the height H′ is preferably 300 nm or more, and more preferably 500 nm or more. Also in this case, the width Wb′ at the bottom is preferably 950 nm or less, and more preferably 900 nm or less.

For the linear convexes that the height H′ is 80 nm or more and 1000 nm or less, and the ratio (Wt′/Wb′) of the width Wt′ at the 97% height of the height to the width Wb′ at the bottom is 0.5 or less, the ratio Wt′/Wb′ is preferably 0.4 or less, and more preferably 0.1 or more and 0.3 or less, from the viewpoint of antibacterial and antifungal properties.

The width Wb′ at the bottom and the width Wt′ at the 97% height of the height are widths shown in horizontal planes being perpendicular to the height direction. When the width of a linear convex varies among cross-sections of the same, it is preferable that the Wt′/Wb′ of each cross-section is in the above range.

From the viewpoint of antibacterial and antifungal properties, the ratio (H′/Wb′) of the height H′ of each linear convex to the width Wb′ at the bottom of the same, is preferably 0.5 or more, more preferably 1.0 or more, and still more preferably 2.0 or more. On the other hand, from the viewpoint of the strength of the linear convexes, the ratio H′/Wb′ is preferably 5.5 or less, more preferably 3.5 or less, and still more preferably 2.5 or less.

In the present disclosure, each linear convex preferably has the following structure: assuming that the linear convex is cut in horizontal planes being perpendicular to the height direction of the same, the cross-sectional area occupancy rate of a material part constituting the linear convex shown in the horizontal cross-sections, gradually increases from the apex of the linear convex to the bottom surface of the same, continuously along the height H′ of the linear convex. More preferably, each linear convex is in such a shape that the cross-sectional area occupancy rate absolutely converges to 0 at the apex.

As the cross-sectional shape of the linear convexes, examples include, but are not limited to, those having vertical cross-sections in polygonal shapes (e.g., a triangle shape, a trapezoidal shape and a pentagonal shape), a pencil shape, a semicircular shape, a semi-elliptical shape, a parabolic shape, a bell shape, etc. From the viewpoint of excellent antibacterial and antifungal properties, the linear convexes are preferably such that the vertical cross-section is in a polygonal or pencil shape, and more preferably such that the vertical cross-section is in a triangle shape. The linear convexes may have the same shape or different shapes.

In the present disclosure, from the viewpoint of increasing antibacterial and antifungal properties, apart not comprising the above-specified linear convexes is typically a substantially flat surface. However, the surface itself of the antibacterial and antifungal article may be curved or ridged. The substantially flat surface means that the surface may have such fine convexes and concaves that the height is 1/100 or less of the lower limit of the above-specified height H′ of the linear convexes (e.g., fine convexes and concaves derived from scratches and raw materials).

For the antibacterial and antifungal article according to the second embodiment, apart of the surface may have convexes that are different from the above-specified linear convexes, as long as the effect of the present disclosure is obtained.

For the antibacterial and antifungal article according to the second embodiment, the area on which the above-specified linear convexes are disposed at the above-specified average two adjacent linear convexes' distance P′_{AVG}, is preferably 70% or more of the total area on which the linear convexes are disposed, more preferably 80% or more, and still more preferably 90% or more.

As the antibacterial and antifungal article according to the second embodiment, examples include, but are not limited to, (i) one comprising a substrate, a convexo-concave layer composed of a different material from the substrate, and the linear convexo-concave shape formed as the surface structure of the convexo-concave layer, (ii) one comprising a substrate and the linear convexes composed of a different material from the substrate and formed on a surface of the substrate, (iii) one comprising the below-described substrate and the linear convexes composed of the same material as the substrate and integrated with the substrate to be formed on a surface of the substrate, the linear convexes constituting the linear convexo-concave shape, and (iv) one having the linear convexo-concave shape formed on a surface of an article and not comprising a substrate. That is, in the second embodiment, the linear convexes constituting the linear convexo-concave shape may be formed on a surface of a convexo-concave layer disposed on a support such as a substrate, may be integrated with a support such as a substrate, or may be directly formed on a surface of a substrate or article. The convexo-concave layer, substrate or article having the linear convexo-concave shape on a surface thereof, may have a monolayer or multilayer structure.

For the antibacterial and antifungal article according to the second embodiment, the material for the linear convexes constituting the linear convexo-concave shape will not be described here since the material may be the same as the material for the projection structure according to the first embodiment. Also, the substrate that the antibacterial and antifungal article according to the present disclosure may comprise, will not be described here since the substrate may be the same as the substrate that the antibacterial and antifungal article according to the first embodiment may comprise.

As with the first embodiment, the antibacterial and antifungal article according to the present disclosure may be a laminate of the antibacterial and antifungal article and an adhesive layer, or it may have a removable protection film on at least a part of the surface thereof.

For the antibacterial and antifungal article according to the second embodiment, the total light transmittance and the contact angle of water with the surface having the linear convexo-concave shape, may be the same as the total light transmittance of the first embodiment and the contact angle of water with the surface having the projection structure of the first embodiment.

### (The Method for Producing the Antibacterial and Antifungal Article According to the Second Embodiment)

The method for producing the antibacterial and antifungal article according to the second embodiment is not particularly limited, as long as it is a method that can produce the above-described antibacterial and antifungal article according to the present disclosure. As the method, examples include, but are not limited to, a shaping method, a photolithography method, a tool cutting method, combinations thereof, an injection molding method, a calendering method and an extrusion molding method. The methods preferred in the first embodiment may be preferably used in the second embodiment.

As the method for producing the antibacterial and antifungal article according to the present disclosure by shaping the convexo-concave shape of the original plate for forming the linear convexo-concave shape, examples include, but are not limited to, the following method: an original plate for forming the linear convexo-concave shape is prepared, which has a convexo-concave-shaped surface having many linear grooves formed thereon (the convexo-concave shape of the convexo-concave-shaped surface corresponds to the linear convexo-concave shape of the antibacterial and antifungal article according to the present disclosure); the convexo-concave-shaped surface of the original plate for forming the linear convexo-concave shape is pressed to a surface of a coating film of the resin composition for forming the linear convexes; and the coating film of the resin composition is cured and then removed from the original plate for forming the linear convexo-concave shape, thereby forming the desired linear convexo-concave shape by shaping. The method for curing the resin composition can be appropriately selected depending on the type and so on of the resin composition.

As the method for forming the convexo-concave shape corresponding to the linear convexo-concave shape on the original plate for forming the linear convexo-concave shape, examples include, but are not limited to, a photolithography method, a laser lithography method, an electron beam lithography method, a tool cutting method and combinations thereof.

In the case of forming the convexo-concave shape corresponding to the linear convexo-concave shape on the original plate for forming the linear convexo-concave shape by any one of the photolithography method, the laser lithography method, the electron beam lithography method and combinations thereof, the convexo-concave shape can be formed by the same method as the method described above under “The method for producing the antibacterial and antifungal article according to the first embodiment”.

As the method for forming the convexo-concave shape on the original plate for forming the linear convexo-concave shape by the tool cutting method, examples include, but are not limited to, the following method: a parent material composed of a metal is cut with a tool, thereby sequentially forming grooves in parallel. The shape of the blade of the tool can be an appropriate shape corresponding to the linear convexo-concave shape to be produced.

In the case of producing the antibacterial and antifungal article according to the present disclosure by the injection or extrusion molding method using the thermoplastic resin composition, for example, the original plate for forming the linear convexo-concave shape produced by the above-described method, can be formed into the shape of the mold of an injection or extrusion molding machine by a desired method and then used. As the method for producing the antibacterial and antifungal article according to the second embodiment by the injection or extrusion molding method, examples include, but are not limited to, the same method as the first embodiment.

### <Applications of the Antibacterial and Antifungal Article>

The antibacterial and antifungal article according to the present disclosure can be used for a variety of applications that are required to provide antibacterial and antifungal properties, and the applications are not particularly limited. As the applications that the antibacterial and antifungal article according to the present disclosure can provide antibacterial and antifungal properties, examples include, but are not limited to, agricultural materials used for plant cultivation facilities (e.g., plastic greenhouses and plant cultivation tanks) and so on; medical devices such as medical tubes (e.g., catheters including cardiovascular catheters, gastrointestinal catheters and urethral catheters), patches for covering a catheter insertion site on the skin, artificial blood vessels, blood bags, medical fluid bags, infusion bags and syringes; dental materials such as mouthpieces; cell culture vessels such as cell culture bags, cell culture plates, cell culture petri dishes, cell culture test tubes, and cell culture flasks; experimental apparatus such as centrifuge tubes; packaging materials such as food and beverage containers; interior materials such as inner walls, ceilings and interior decorations used for rooms and spaces equipped with plumbing systems such as bath, sink, laundry, kitchen and toilet installations (including modular bathrooms) and rooms and spaces next to plumbing systems, such as undressing rooms, drying areas and dining rooms; exterior materials such as gates, fences, exterior walls and carports; air-conditioning machines such as air conditioners and air purifiers; home electrical appliances such as refrigerators, washing machines, telephones and cleaners; cooking devices such as microwave ovens and rice cookers; medical facilities such as medical equipment; and school facilities such as office machines and other electronics. Examples also include antibacterial and antifungal filters used in these various kinds of devices, and protection films (for electronic display, touch panel, etc.), casings and window films of these various kinds of articles. The antibacterial and antifungal article according to the present disclosure may be in such a form that it has the projection structure or linear convexo-concave shape on the inner surface, outer surface or both surfaces. Since the antibacterial and antifungal article according to the present disclosure can keep antibacterial and antifungal properties for a long period of time, it can be preferably used for parts out of the reach of everyone in various kinds of articles, such as carport roofing materials and antibacterial filters, etc., installed in the various kinds of devices. Also, the antibacterial and antifungal article according to the present disclosure can be particularly preferably used for applications required to reduce biofilm formation. As such applications, examples include, but are not limited to, the above-described medical devices and dental materials, the above-mentioned interior materials used for plumbing systems, cell culture vessels, experimental apparatus, and food and beverage containers and packaging materials.

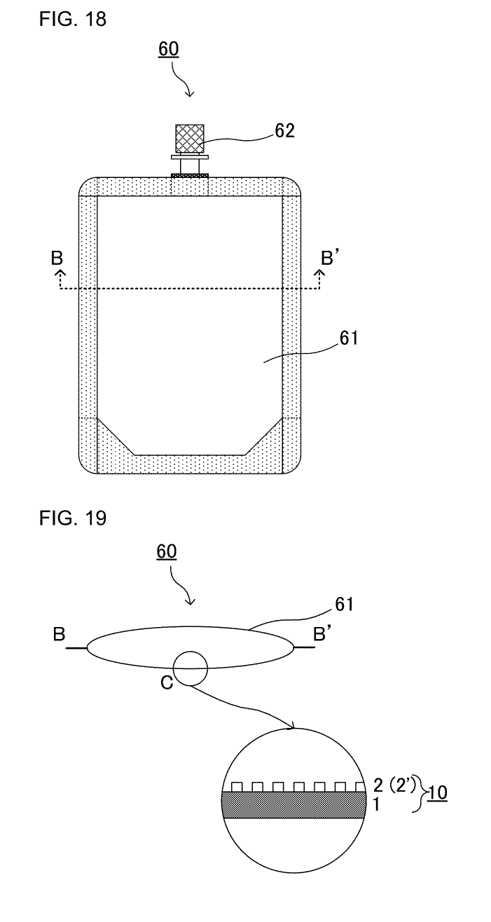

Examples of the above-mentioned containers and packaging materials will be described with reference to examples. FIG. 18 is a schematic view of an example of the mode of use of the antibacterial and antifungal article according to an embodiment. FIG. 19 is a schematic cross-sectional view of an example of a B-B′ cross-sectional view shown in FIG. 18. FIG. 19 also shows an enlarged view of a part C. FIGS. 18 and 19 show an example of a container for storing a liquid material, that is, an example of a so-called pouch container. A container 60 shown in FIGS. 18 and 19 has such a shape that two packaging materials 61 are stacked and attached to each other at their peripheral edges. In order to increase the volume of the container, three packaging materials 61 are attached at the bottom of the container. Also, an outlet port 62 that can be sealed, is provided at the top of the container. As shown by the example in FIG. 19, the B-B′ cross-section shows a space for housing a liquid material formed between the two packaging materials 61. For example, the antibacterial and antifungal article according to the disclosed embodiment can be disposed inside the space for housing a liquid material. That is, the antibacterial and antifungal article according to the disclosed embodiment can provide the inside of the space for housing a liquid material with the projection structure or convexes and concaves having the linear convexo-concave shape; therefore, propagation of bacteria or fungi in the liquid material can be reduced (see C in FIG. 19). The projection structure or the linear convexo-concave shape can be disposed on the outer surface of the packaging material 61 (not shown).

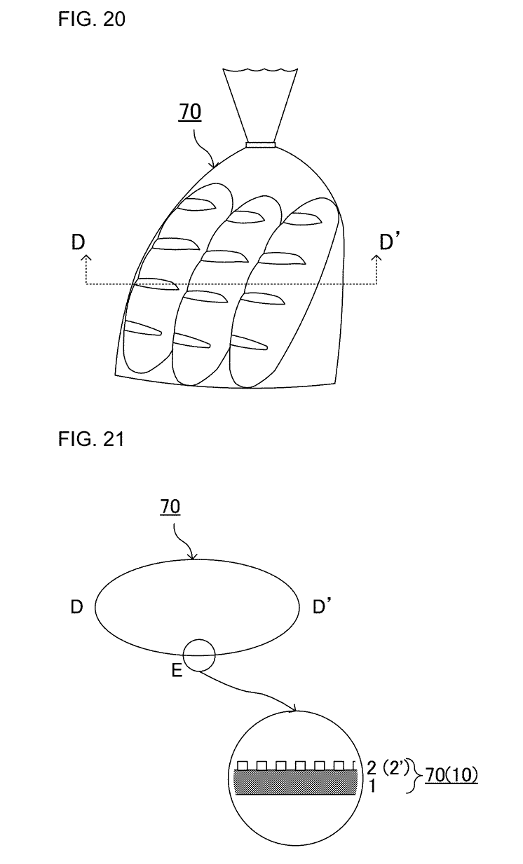

FIG. 20 is a schematic view of another example of the mode of use of the antibacterial and antifungal article according to an embodiment. FIG. 21 is a schematic cross-sectional view of an example of a D-D′ cross-sectional view shown in FIG. 20. FIG. 21 also shows an enlarged view of a part E. FIGS. 20 and 21 show an example of a packaging material 70 for storing foods such as bread and vegetables, that is, an example of a so-called wrapping film. As shown in FIG. 21, for the packaging material 70, the inner surface of the space for housing foods is a surface having the projection group. In general, when an object such as food is housed in a packaging material, bacteria or fungi start to propagate from a part of the object in contact with the packaging material and then cover the object. By use of the antibacterial and antifungal article according to the disclosed embodiment as a packaging material, the propagation of bacteria or fungi on the surface of the packaging material is reduced, so that the propagation of bacteria or fungi on the part of the housed object (such as food) in contact with the packaging material, can be reduced. Therefore, the propagation of bacteria or fungi can be reduced all over the housed object. In the case of using the antibacterial and antifungal article according to the disclosed embodiment as a packaging material, from the viewpoint of increasing antibacterial and antifungal effects, it is preferable that at least a part of the inner surface is a surface having the projection structure or the linear convexo-concave shape, and it is more preferable that the inner surface of the space for housing the object is a surface having the projection structure or the linear convexo-concave shape.

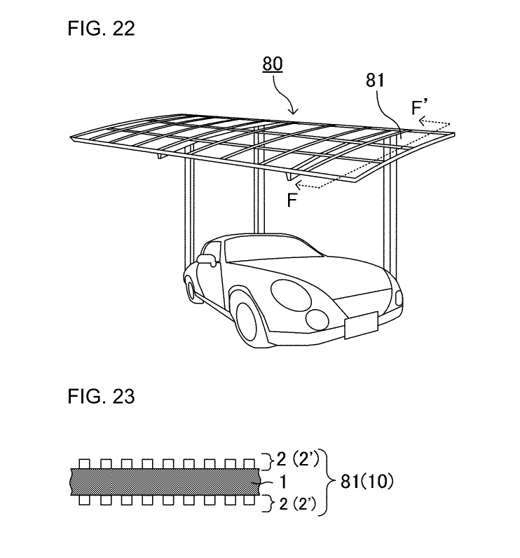

An example of the exterior materials will be described in detail with reference to figures. FIG. 22 is a schematic view of another example of the mode of use of the antibacterial and antifungal article according to an embodiment. FIG. 23 is a schematic cross-sectional view of an example of an enlarged part of an F-F′ cross section shown in FIG. 22. FIGS. 22 and 23 show an example of the case of using the antibacterial and antifungal article according to the disclosed embodiment as a roofing material 81 for a carport 80. As shown in FIG. 23, both surfaces of the roofing material 81 for the carport 80, are surfaces having the projection structure or the linear convexo-concave shape.

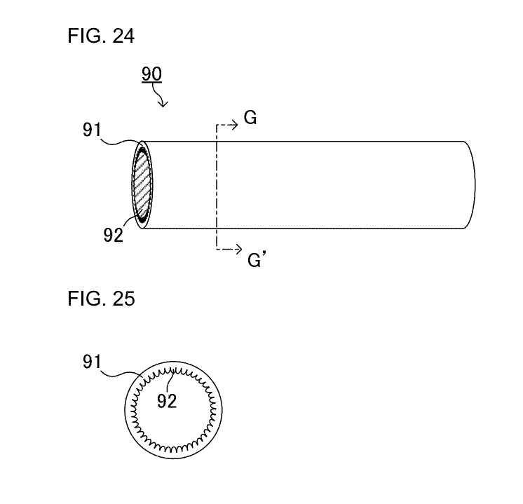

The antibacterial and antifungal article according to the disclosed embodiment can be preferably used for medical applications and can be preferably used as antibacterial and antifungal medical device. At least apart of the antibacterial and antifungal medical device according to the disclosed embodiments, comprises the antibacterial and antifungal article according to the disclosed embodiment. For example, apart of the medical device may be composed of the antibacterial and antifungal article according to the disclosed embodiments, or the medical device itself may be the antibacterial and antifungal article according to the disclosed embodiment. Also, the antibacterial and antifungal medical device according to the disclosed embodiment may be such that the antibacterial and antifungal article according to the disclosed embodiment in a sheet or film shape is attached to at least a part of the surface of the medical device. FIG. 24 is a schematic perspective view of an example of the mode of use of antibacterial and antifungal medical device that is the antibacterial and antifungal article according to an embodiment. FIG. 25 is a schematic cross-sectional view of an example of a G-G′ cross section shown in FIG. 24. FIGS. 24 and 25 show an example of the case of using the antibacterial and antifungal medical device according to the disclosed embodiment as a medical tube 90, and the inner surface of a support 91 in a cylindrical tube shape has a projection structure or linear convexo-concave shape 92. In FIG. 24, the projection structure or linear convexo-concave shape 92 is marked with diagonal lines.

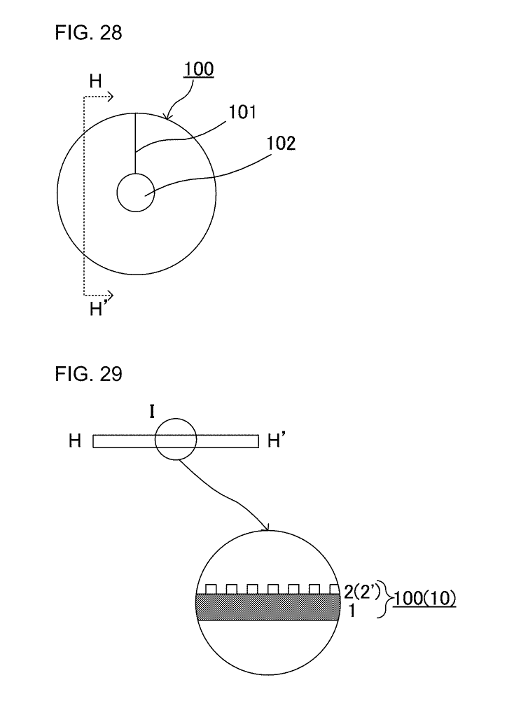

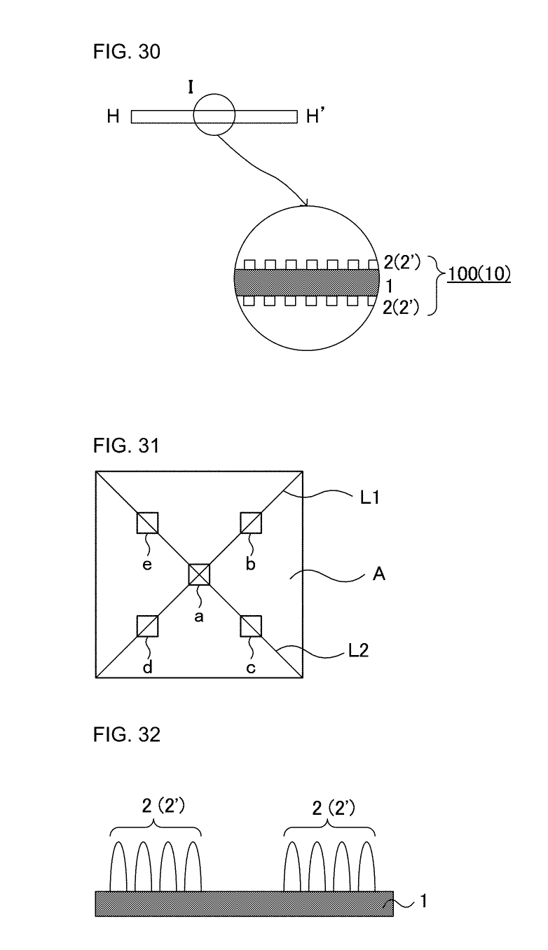

FIG. 28 is a schematic front view of another example of the mode of use of the antibacterial and antifungal medical device that is an antibacterial and antifungal article according to an embodiment. FIG. 29 is a schematic cross-sectional view of an example of an H-H′ cross section shown in FIG. 28. FIG. 30 is a schematic cross-sectional view of another example of the H-H′ cross section shown in FIG. 28. FIGS. 29 and 30 show enlarged views of a part I. FIG. 28 is the case of using the antibacterial and antifungal medical device according to the disclosed embodiment as a medical patch 100 for covering a catheter insertion site on the skin. As shown in FIG. 29, the projection structure 2 or the linear convexo-concave shape 2′ may be disposed on one surface of the medical patch 100. As shown in FIG. 30, the projection structure 2 or the linear convexo-concave shape 2′ may be disposed on both surfaces of the medical patch 100. The medical patch 100 has a slit 101 and a catheter insertion site 102 which is a hole for inserting a catheter. When the antibacterial and antifungal medical device according to the disclosed embodiment is used as a medical patch, the medical patch may have the projection structure or the linear convexo-concave shape on at least a part of the surface thereof, or the medical patch may have the projection structure or the linear convexo-concave shape on the whole of one surface or each of both surfaces thereof.

The antibacterial and antifungal article according to the present disclosure may be preferably used for agricultural applications and may be preferably used as an antibacterial and antifungal agricultural material. A part of the antibacterial and antifungal agricultural material comprises the antibacterial and antifungal article according to the present disclosure. As with the antibacterial and antifungal medical device, a part of the antibacterial and antifungal agricultural material may comprise the antibacterial and antifungal article according to the present disclosure, or the agricultural material itself may be the antibacterial and antifungal article according to the present disclosure. The antibacterial and antifungal agricultural material according to the present disclosure can reduce the propagation of fungi and bacteria, which are called plant pathogens, can stably grow crops, and can increase yields. As the plant pathogens, examples include, but are not limited to, those described in “All about hydroponics” edited by Japan Greenhouse Horticulture Association and Hydroponic Society of Japan. It was found that the antibacterial and antifungal agricultural material according to the present disclosure has high antifungal properties against fungi such as Pythium and Fusarium.

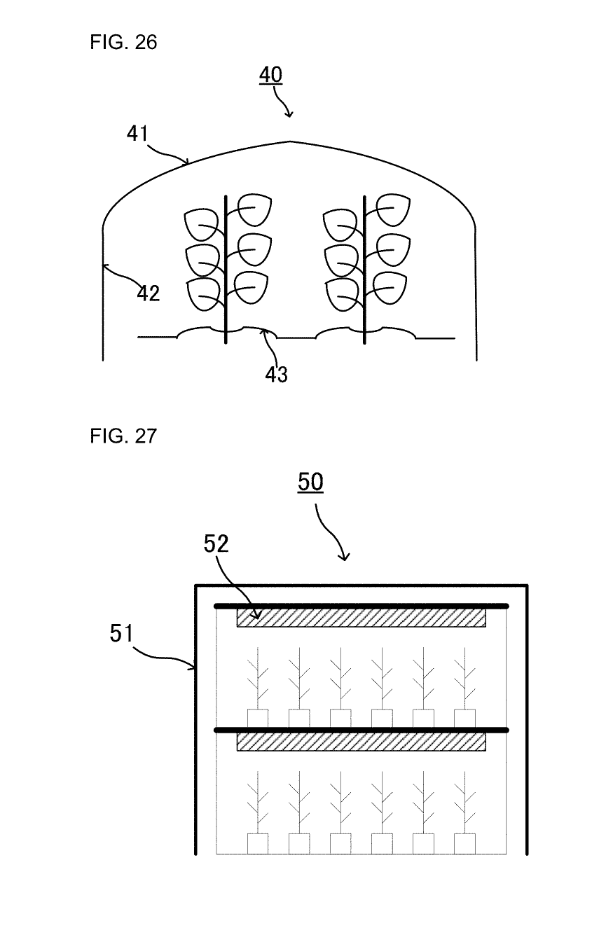

The mode of use of the antibacterial and antifungal agricultural material according to the present disclosure will be described with reference to figures. FIG. 26 is a schematic view of an example of the mode of use of the antibacterial and antifungal agricultural material according to an embodiment. More specifically, it is a schematic cross-sectional view of a plastic greenhouse 40. For example, the antibacterial and antifungal agricultural material according to the disclosed embodiment may be disposed at the inner surface side of a ceiling 41 or a wall 42, or it may be disposed on a surface of a reflective sheet disposed on a soil surface 43. Also, the antibacterial and antifungal agricultural material according to the disclosed embodiment may be sheet-shaped or plate-shaped materials that can constitute the ceiling 41 or the wall 42, or may be film-shaped materials that are attached to the inner surface side of the ceiling 41 or the wall 42 for use.

FIG. 27 is a schematic view of another example of the mode of use of the antibacterial and antifungal agricultural material according to an embodiment. More specifically, it is a schematic cross-sectional view of an example of a plant cultivation unit 50 (also referred to as LED house) in plant factory cultivation. The plant cultivation unit shown in FIG. 27 is such that a light source 52 (such as LED light source) is disposed at the top board side of a shelf or at the top board sides of the multi-layered shelves. The shelf (shelves) is provided with a reflective sheet 51 for efficient use of light from the light source and control of temperature and humidity conditions. For example, the antibacterial and antifungal agricultural material according to the disclosed embodiment may be disposed at the inner surface side of the reflective sheet 51 or may be provided to a shelf board or the top board of a shelf.

By use of the antibacterial and antifungal agricultural material according to the disclosed embodiment, the amount of pesticides used (e.g., antibacterial and antifungal agents) can be reduced; the yield of crops can be increased; and stable production of crops can be achieved.

### EXAMPLES

Hereinafter, the disclosed embodiments will be described in detail, by way of examples. The disclosed embodiments are not limited by the following descriptions. For each projection, the height H, the width Wt at the 97% height, and the width Wb at the bottom were measured by a SEM and cross-section profile analysis using a laser microscope (product name: LEXT OLS4100, manufactured by Olympus Corporation). For each linear convex, the height H′, the width Wt′ at the 97% height, and the width Wb′ at the bottom were measured in the same manner.

### Production Example 1: Production of an Original Plate A for Forming a Projection Structure

A roll-pressed aluminum plate with a purity of 99.50%, was polished so that the surface had a convexo-concave shape with a 10-point average roughness Rz of 30 nm and a period of 1 μm. Then, in an electrolyte (0.04 M oxalic acid aqueous solution), anodization was carried out at a formation voltage of 20 V and a temperature of 20° C. for 120 seconds. Next, as a first etching treatment, an etching treatment was carried out for 60 seconds in the electrolyte used in the anodization. Then, as a second etching treatment, pore diameter regulation was carried out in a 1.0 M phosphoric acid aqueous solution for 150 seconds. In addition, these processes were repeated a total of five times in series. Therefore, an anodized aluminum layer was formed, which is such a layer that a fine convexo-concave shape is formed on the aluminum substrate. Finally, a fluorine-based release agent was applied to the anodized aluminum layer, and the excess release agent was removed from the layer by washing, thereby obtaining the original plate A for forming the projection structure. The fine convexo-concave shape formed on the aluminum layer was such a shape that many fine pores are densely formed at an average interval of 200 nm and the pore diameter gradually decreases in the depth direction.

### Production Example 2: Production of an Original Plate B for Forming a Projection Structure

The original plate B for forming the projection structure was obtained in the same manner as the production of the original plate A, except that the formation voltage was changed to 25 V, and the second etching treatment time was changed to 180 seconds. The fine convexo-concave shape formed on the aluminum layer was such a shape that many fine pores are densely formed at an average interval of 100 nm and the pore diameter gradually decreases in the depth direction.

### Production Example 3: Production of an Original Plate C for Forming a Projection Structure

A stainless-steel plate was subjected to blasting so that the arithmetic mean surface roughness (hereinafter referred to as Sa) measured by three-dimensional surface roughness measurement was 0.2 μm. The stainless-steel plate was subjected to electrolytic chromium plating in the following conditions to obtain the original plate C for forming the projection structure, the original plate having many cone-shaped convex projections on a surface thereof.

### <Electrolytic Chromium Plating Conditions>

In a plating bath of the following composition, using a graphite electrode as an anode, a black chromium plating film was formed on the stainless-steel plate by electrolytic plating, decreasing current density by 2.0 A/dm² every one minute from 80 A/dm² to 20 A/dm².

<<The Composition of the Plating Bath>>
Chromium chloride: 200 g/dm³ (0.75 mol/dm³)
Ammonium chloride: 30 g/dm³ (0.56 mol/dm³)
Oxalic acid: 3 g/dm³ (0.024 mol/dm³)
Barium carbonate: 5 g/dm³ (0.025 mol/dm³)
Boric acid: 30 g/dm³ (0.49 mol/dm³)
Barium fluoride: 10 g/dm³ (0.057 mol/dm³)

### Production Example 4: Production of an Original Plate D for Forming a Linear Convexo-Concave Shape

A silicon wafer with a thickness of 600 μm was used as a substrate. Next, a surface of the silicon wafer substrate was thermally oxidized to forma silicon oxide film that serves as a mask for silicon etching. Then, a resist pattern was formed by an electron beam lithography method or a photolithography method. The silicon oxide film exposed at the openings of the resist pattern was removed by a dry etching method. Then, the resist was removed by O₂ plasma asking, thereby forming an etching mask pattern corresponding to apart to be formed. For the etching mask pattern, the line width was 100 nm; and the pitch was 400 nm; and the mask lines were disposed in parallel at regular intervals.

Next, the silicon wafer was subjected to a crystal anisotropic etching treatment. In particular, the silicon wafer was immersed in a tetramethylammonium hydroxide solution at a concentration of 25% and a temperature of 23° C., thereby producing the original plate D for forming the linear convexo-concave shape, the original plate having linear prism-shaped grooves with a depth of about 200 nm, a line width of about 300 nm, and a pitch of about 400 nm.

### Production Example 5: Production of an Original Plate E for Forming a Projection Structure

A silicon wafer with a thickness of 600 μm was used as a substrate. Next, a surface of the silicon wafer substrate was thermally oxidized to form a silicon oxide film that serves as a mask for silicon etching. Then, a resist pattern was formed by an electron beam lithography method or a photolithography method. The silicon oxide film exposed at the openings of the resist pattern was removed by a dry etching method. Then, the resist was removed by O₂ plasma asking, thereby forming an etching mask pattern corresponding to a part to be formed. For the etching mask pattern, the width was 50 nm; the pitch was 350 nm; and the mask lines were disposed in a grid pattern.

Next, the silicon wafer was subjected to a crystal anisotropic etching treatment. In particular, the silicon wafer was immersed in a tetramethylammonium hydroxide solution at a concentration of 25% and a temperature of 23° C., thereby producing the original plate E for forming the projection structure, the original plate having pyramid-shaped pores with a depth of about 200 nm, a bottom width of about 300 nm, and a pitch of about 350 nm.

### Production Example 6: Production of an Original Plate F for Forming a Linear Convexo-Concave Shape

First, a substrate comprising a base and a convex structure protruding from one surface of the base, was prepared. An electron beam-sensitive resist film was formed on the top surface (a pattern formed surface) of the convex structure. Next, using an electron beam lithography system, a pattern image for forming the linear convexo-concave shape was written on the electron beam-sensitive resist film. Then, a resist pattern for forming the linear convexo-concave shape was formed on the top surface (the pattern formed surface) of the convex structure by development using a predetermined developer. Then, dry etching was carried out using the resist pattern as a mask, thereby producing the original plate F for forming the linear convexo-concave shape, the original plate having the linear convexo-concave shape (pitch 100 nm) formed on the pattern formed surface of the convex structure. The original plate F was an imprint mold.

### Comparative Production Example 1: Production of an Original Plate G for Forming a Projection Structure

As a hard mask material layer, a thin chromium film (thickness 15 nm) was formed on a quartz substrate (thickness 6.35 mm) by a sputtering method. Then, a commercially-available, electron beam-sensitive resist was applied onto the thin chromium film. Next, the quartz substrate was placed on a stage inside a commercially-available electron beam lithography system, and the applied resist was exposed to electron beam irradiation, thereby forming a patterned latent image on the resist.

Next, the resist was developed to form a resist pattern. Using the resist pattern as an etching mask, the hard mask material layer was subjected to dry-etching to form a chromium hard mask. Then, using the hard mask as an etching mask, the quartz substrate was subjected to dry etching, thereby producing the original plate G for forming the projection structure, the original plate having a fine concave pattern with a depth of 200 nm, a pitch of 400 nm and a width of 200 nm.

### Comparative Production Example 2: Production of an Original Plate H for Forming a Linear Convexo-Concave Shape

First, a diamond tool having fine concaves and comprising monocrystalline diamond was prepared, the fine concaves being stripe-shaped concaves corresponding convexo-concave grooves. Using the diamond tool, a surface of a metal substrate was subjected to cutting, thereby forming the original plate H for forming the linear convexo-concave shape. The diamond tool was produced by the method described in Japanese Patent Application Laid-Open No. 2013-146795. As the metal substrate, a roll-pressed aluminum plate with a purity of 99.50% was used.

### Production Example 7: Production of an Original Plate I for Forming a Linear Convexo-Concave Shape

A silicon wafer with a thickness of 600 μm was used as a substrate. Next, a surface of the silicon wafer substrate was thermally oxidized to form a silicon oxide film that serves as a mask for silicon etching. Then, a resist pattern was formed by a photolithography method. The silicon oxide film exposed at the openings of the resist pattern was removed by a dry etching method. Then, the resist was removed by O₂ plasma asking, thereby forming an etching mask pattern corresponding to apart to be formed. For the etching mask pattern, the line width was 500 nm; the pitch was 800 nm; and the mask lines were disposed in parallel at regular intervals.

Next, the silicon wafer was subjected to a crystal anisotropic etching treatment. In particular, the silicon wafer was immersed in a tetramethylammonium hydroxide solution at a concentration of 25% and a temperature of 23° C., thereby producing the original plate I for forming the linear convexo-concave shape, the original plate having linear prism-shaped grooves with a depth of about 900 nm, a line width of about 500 nm, and a pitch of about 800 nm.

### Production Examples 8 to 28: Production of Original Plates A2 to A22 for Forming Projection Structures

The original plates A2 to A22 for forming the projection structures were obtained in the same manner as Production Example 1, except that the formation voltage in the anodization treatment was appropriately controlled; the first etching treatment time and the second etching treatment time were appropriately controlled; and convexo-concave shapes corresponding to projection structures with average intervals and heights shown in Table 2, were formed.

### Example 1

A resin composition for forming a projection structure, which is a resin composition of the following composition, was applied to the original plate A for forming the projection structure to a thickness of 20 μm so that a surface of the original plate A was covered with the resin composition. As a transparent substrate, a triacetyl cellulose film with a thickness of 80 μm (product name: T80SZ, manufactured by: FUJIFILM Corporation) was attached thereon. A laminate thus obtained was pressed by a rubber roller at a load of 10 N/cm². After confirming that the composition was uniformly applied to the original plate A for forming the projection structure, ultraviolet rays were applied from the transparent substrate side at 2000 mJ/cm² to cure the resin composition for forming the projection structure, thereby producing a convexo-concave layer having the projection structure on the transparent substrate. Then, the transparent substrate and the convexo-concave layer (a cured product of the resin composition for forming the projection structure) were removed from the original plate A for forming the projection structure, thereby obtaining an antibacterial and antifungal article.

For the antibacterial and antifungal article of Example 1, such projections were 88% of all projections, that the average P_{AVG} of two adjacent projections' distances was 200 nm; the height H was 330 nm; the ratio (Wt/Wb) of the width Wt at the 97% height of the height to the width Wb at the bottom was 0.30.

FIG. 6 is a photograph of a cross section of the antibacterial and antifungal article of Example 1 taken by SEM. As shown in FIG. 6, for the projections of the projection structure of the antibacterial and antifungal article of Example 1, the vertical cross section was in a triangle or parabolic shape.

### <The Composition of the Resin Composition for Forming the Projection Structure>

The resin composition for forming the projection structure was prepared by dissolving the following components in 200 parts by mass of ethyl acetate.
Dipentaerythritol hexaacrylate (DPHA): 23 parts by mass
ARONIX M-260 (product name, polyethylene glycol diacrylate manufactured by TOAGOSEI Co., Ltd.): 72 parts by mass
Hydroxyethyl acrylate: 5 parts by mass
Photocuring agent (product name: Lucirin TPO, manufactured by: BASF): 3 parts by mass

### Example 2

An antibacterial and antifungal article was obtained in the same manner as Example 1, except that the original plate B for forming the projection structure was used in place of the original plate A for forming the projection structure.

For the antibacterial and antifungal article of Example 2, such projections were 98% of all projections, that the average P_{AVG} of two adjacent projections' distances was 100 nm; the height H was 235 nm; and the ratio (Wt/Wb) of the width Wt at the 97% height of the height to the width Wb at the bottom was 0.28.

FIG. 7 is a photograph of a cross section of the antibacterial and antifungal article of Example 2 taken by SEM. As shown in FIG. 7, for the projections of the projection structure of the antibacterial and antifungal article of Example 2, the vertical cross section in a parabolic shape.

### Example 3

An antibacterial and antifungal article was obtained in the same manner as Example 1, except that the original plate C for forming the projection structure was used in place of the original plate A for forming the projection structure.

For the antibacterial and antifungal article of Example 3, such projections were 65% of all projections, that the average P_{AVG} of two adjacent projections' distances was 375 nm; the height H was 908 nm; and the ratio (Wt/Wb) of the width Wt at the 97% height of the height to the width Wb at the bottom was 0.3.

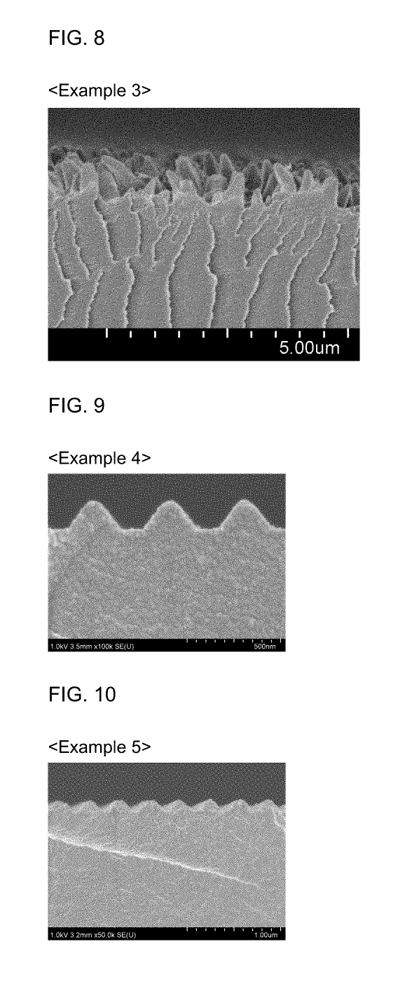

FIG. 8 is a photograph of a cross section of the antibacterial and antifungal article of Example 3 taken by SEM. As shown in FIG. 8, for the projections of the projection structure of the antibacterial and antifungal article of Example 3, the vertical cross section was in a triangle or parabolic shape.

### Example 4

An antibacterial and antifungal article was obtained in the same manner as Example 1, except that the original plate D for forming the linear convexo-concave shape was used in place of the original plate A for forming the projection structure.

For the antibacterial and antifungal article of Example 4, such linear convexes were 99% of all linear convexes, that the average P′_{AVG} of two adjacent linear convexes' distances was 400 nm; the height H′ was 144 nm; and the ratio (Wt′/Wb′) of the width Wt′ at the 97% height of the height to the width Wb′ at the bottom was 0.22.

FIG. 9 is a photograph of a cross section of the antibacterial and antifungal article of Example 4 taken by SEM. As shown in FIG. 9, for the linear convexes of the linear convexo-concave shape of the antibacterial and antifungal article of Example 4, the vertical cross section was in a triangle shape.

### Example 5

An antibacterial and antifungal article was obtained in the same manner as Example 1, except that the original plate E for forming the projection structure was used in place of the original plate A for forming the projection structure.

For the antibacterial and antifungal article of Example 5, such linear convexes were 99% of all linear convexes, that the average P_{AVG} of two adjacent projections' distances was 350 nm; the height H was 139 nm; and the ratio (Wt/Wb) of the width Wt at the 97% height of the height to the width Wb at the bottom was 0.12.

FIG. 10 is a photograph of a cross section of the antibacterial and antifungal article of Example 5 taken by SEM. As shown in FIG. 10, for the projections of the projection structure of the antibacterial and antifungal article of Example 5, the vertical cross section was in a triangle shape.

### Example 6

An antibacterial and antifungal article was obtained in the same manner as Example 1, except that the original plate F for forming the linear convexo-concave shape was used in place of the original plate A for forming the projection structure.

For the antibacterial and antifungal article of Example 6, such linear convexes were 86% of all linear convexes, that the average P′_{AVG} of two adjacent linear convexes' distances was 100 nm; the height H′ was 126 nm; and the ratio (Wt′/Wb′) of the width Wt′ at the 97% height of the height to the width Wb′ at the bottom was 0.40.

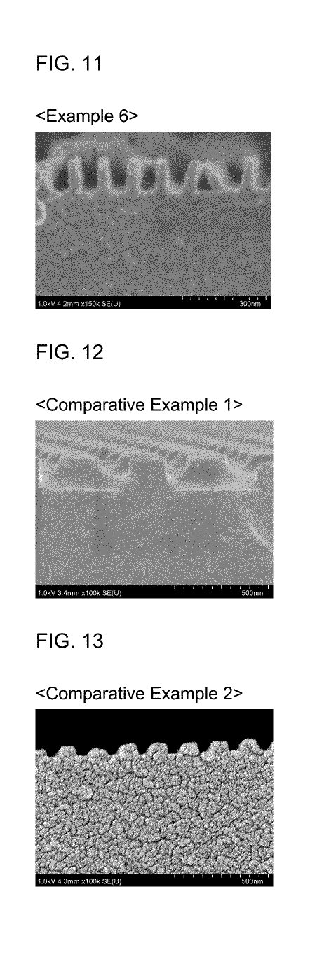

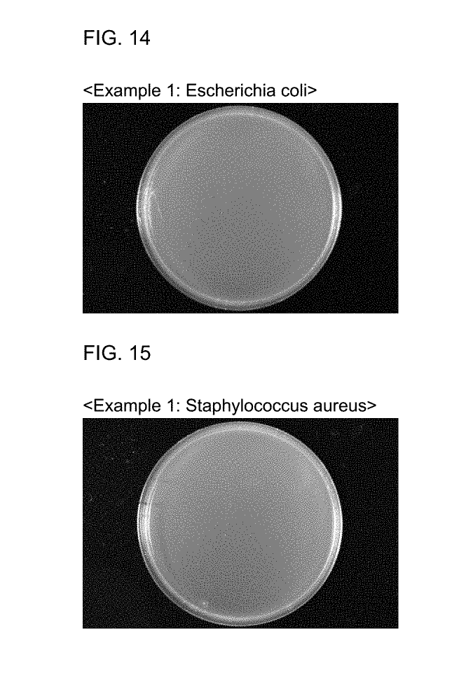

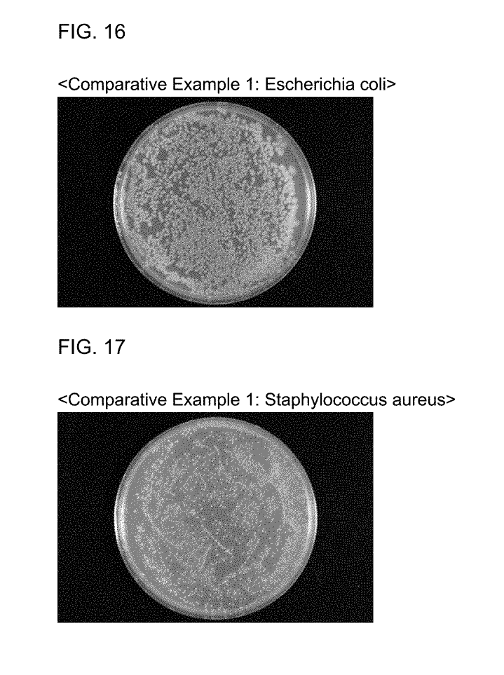

FIG. 11 shows a photograph of a cross section of the antibacterial and antifungal article of Example 6 taken by SEM. As shown in FIG. 11, for the linear convexes of the linear convexo-concave shape of the antibacterial and antifungal article of Example 6, the vertical cross section was in tapered square shape.

### Comparative Example 1

A comparative article was obtained in the same manner as Example 1, except that the original plate G for forming the projection structure was used in place of the original plate A for forming the projection structure.

For the comparative article of Comparative Example 1, the average P_{AVG} of two adjacent projections' distances was 400 nm; the height H of the projections was 179 nm; and the ratio (Wt/Wb) of the width Wt at the 97% height of the height to the width Wb at the bottom was 0.56.

### Comparative Example 2

A comparative article was obtained in the same manner as Example 1, except that the original plate H for forming the linear convexo-concave shape was used in place of the original plate A for forming the projection structure.

For the comparative article of Comparative Example 2, the average P′_{AVG} of two adjacent linear convexes' distances was 180 nm; the height H′ of the linear convexes was 62 nm; and the ratio (Wt′/Wb′) of the width Wt′ at the 97% height of the height to the width Wb′ at the bottom was 0.55.

### Comparative Example 3

A comparative article was obtained as follows: the resin composition for forming the projection structure was applied onto a substrate (material: PET, thickness: 100 μm, product name: Lumirror U34, manufactured by: Toray Industries, Inc.) so that the thickness of the resin composition was 20 μm when cured. Moreover, ultraviolet rays were applied from the substrate side at 2000 mJ/cm² to cure the resin composition, thereby obtaining the comparative article of Comparative Example 3.

### Examples 7 to 27

Antibacterial and antifungal articles were obtained in the same manner as Example 1, except that the original plates A2 to A22 for forming the projection structures were used in place of the original plate A for forming the projection structure.

For the antibacterial and antifungal articles of Examples 7 to 27, the average P_{AVG} of two adjacent projections' distances, the height H, the width Wt at the 97% height of the height, and the width Wb at the bottom are shown in Table 2. For each of the antibacterial and antifungal articles of Examples 7 to 27, projections in the size shown in Table 2 were 70 to 95% of all projections.

### <Antibacterial Evaluation 1>

### (Production of Test Bacterial Solutions)

Of the test bacteria listed below, Staphylococcus aureus was inoculated into a nutrient agar medium, cultured at 35±1° C. for 18 hours, cultured again at 35±1° C. for 18 hours, and adjusted to 2.5×10⁵ to 10×10⁵/mL using a nutrient broth diluted 100 times ( 1/100 NB). The resulting product was used as a test bacterial solution. Also, Escherichia coli was subjected to the same procedure to prepare another test bacterial solution.

[Test Bacteria]

Staphylococcus aureus NBRC12732

Escherichia coli NBRC3972

### (Production of Test Samples)

The antibacterial and antifungal articles obtained in Examples 1 to 27 and Comparative Examples 1 to 3 were wiped with ethanol for disinfection and used as test samples. A sterile PET film (product name: A4100, manufactured by: Toyobo Co., Ltd.) was cut into a 5 cm-square piece and used as a control.

### (Inoculation and Culture of the Test Bacterial Solutions)

The test bacterial solutions were inoculated into the test samples (including the control), covered with films, placed in petri dishes, and then cultured for 24 hours under the following conditions:

Temperature: 35±1° C.

Relative humidity: 90% or more.

### (Determination of Viable Bacteria Number)

The control was washed out with a SCDLP culture medium just after the inoculation and 24 hours after the culture, thereby obtaining test solutions. Each test sample was washed with a SCDLP culture medium 24 hours after the culture, thereby obtaining a test solution. The resulting test solutions were diluted to obtain 10-fold diluted solutions. Each diluted solution was inoculated into a SCDLP agar medium and cultured at 35±1° C. for 48 hours. After the culture, the number of colonies thus formed was counted and converted into the number of viable bacteria.

Tables 1 and 2 show the evaluation results of antibacterial activity values calculated by the following formula:

Antibacterial activity value=log (the number of viable bacteria on the control)−log (the number of viable bacteria on the antibacterial article of each example or comparative example)

When the antibacterial activity value is 2.0 or more, the article is determined to have antibacterial effects.

TABLE 1

| | | | | | | Antibacterial activity values | |
|---|---|---|---|---|---|---|---|
| | H | Wt (H97%) | Wb | | | | Staphylococcus |
| | (nm) | (nm) | (nm) | Wt/Wb | H/Wb | Escherichia coli | aureus |
| Example 1 | 330 | 45 | 150 | 0.30 | 2.20 | 8.9 | 7.3 |
| Example 2 | 235 | 37 | 130 | 0.28 | 1.81 | 3.5 | 7.3 |
| Example 3 | 908 | 90 | 300 | 0.30 | 3.03 | 3.8 | 6.1 |
| Example 4 | 144 | 64 | 286 | 0.22 | 0.50 | 3.5 | 5.6 |
| Example 5 | 139 | 35 | 290 | 0.12 | 0.48 | 3.2 | 5.6 |
| Example 6 | 126 | 21 | 53 | 0.40 | 2.38 | 2.3 | 4.4 |
| Comparative | 179 | 178 | 316 | 0.56 | 0.57 | 0.2 | 0.1 |
| Example 1 | | | | | | | |
| Comparative | 62 | 57 | 104 | 0.55 | 0.60 | 0.4 | 2.0 |
| Example 2 | | | | | | | |
| Comparative | 0 | — | — | — | — | 0.1 | 0.1 |
| Example 3 | | | | | | | |

TABLE 2

| | | | | | | | Antibacterial activity values | |
|---|---|---|---|---|---|---|---|---|
| | P_{AVG} | H | Wt (H97%) | Wb | | | | Staphylococcus |
| | (nm) | (nm) | (nm) | (nm) | Wt/Wb | H/Wb | Escherichia coli | aureus |
| Example 7 | 150 | 201 | 76 | 152 | 0.5 | 1.32 | 3.5 | 5.5 |
| Example 8 | 150 | 203 | 52 | 153 | 0.34 | 1.33 | 4.8 | 6.0 |
| Example 9 | 180 | 202 | 33 | 155 | 0.21 | 1.3 | 5.8 | 6.2 |
| Example 10 | 155 | 206 | 17 | 149 | 0.11 | 1.38 | 6.3 | 6.4 |
| Example 11 | 450 | 999 | 104 | 518 | 0.2 | 1.93 | 3.8 | 5.9 |
| Example 12 | 400 | 980 | 51 | 492 | 0.1 | 1.99 | 4.3 | 6.0 |
| Example 13 | 100 | 102 | 48 | 101 | 0.48 | 1.01 | 4.5 | 4.8 |
| Example 14 | 150 | 107 | 14 | 105 | 0.13 | 1.02 | 6.3 | 6.7 |
| Example 15 | 200 | 503 | 82 | 254 | 0.32 | 1.98 | 4.0 | 5.8 |
| Example 16 | 250 | 508 | 44 | 249 | 0.18 | 2.04 | 4.9 | 6.1 |
| Example 17 | 200 | 501 | 25 | 150 | 0.17 | 3.34 | 6.9 | 6.4 |
| Example 18 | 200 | 505 | 33 | 205 | 0.16 | 2.46 | 6.2 | 5.9 |
| Example 19 | 120 | 508 | 23 | 98 | 0.23 | 5.18 | 6.5 | 6.0 |
| Example 20 | 300 | 755 | 47 | 410 | 0.11 | 1.84 | 5.8 | 6.4 |
| Example 21 | 100 | 155 | 22 | 69 | 0.32 | 2.25 | 6.0 | 6.9 |
| Example 22 | 150 | 253 | 30 | 200 | 0.15 | 1.27 | 6.7 | 6.9 |
| Example 23 | 180 | 300 | 38 | 152 | 0.25 | 1.97 | 4.6 | 4.2 |
| Example 24 | 150 | 300 | 28 | 122 | 0.23 | 2.46 | 5.2 | 4.6 |
| Example 25 | 200 | 300 | 50 | 205 | 0.24 | 1.46 | 3.8 | 4.1 |
| Example 26 | 150 | 400 | 55 | 197 | 0.28 | 2.03 | 5.3 | 5.0 |
| Example 27 | 100 | 400 | 32 | 155 | 0.21 | 2.58 | 6.1 | 5.7 |

For Example 4, Example 6 and Comparative Example 2, H, Wt and Wb in Table 1 are understood as H′, Wt′ and Wb′, respectively.

### Example 28

An antibacterial and antifungal article was obtained in the same manner as Example 1, except that the original plate I for forming the linear convexo-concave shape was used in place of the original plate A for forming the projection structure.

For the antibacterial and antifungal article of Example 28, such linear convexes were 90% of all linear convexes, that the average P′_{AVG} of two adjacent linear convexes' distances was 800 nm; the height H′ was 900 nm; the width Wt′ at the 97% height of the height was 225 nm; the width Wb′ at the bottom was 500 nm; Wt′/Wb′ was 0.45; and H′/Wb′ was 1.8.

### <Antifungal Evaluation 1>

The antibacterial and antifungal articles of Examples 1, 6 and 28 and the articles of Comparative Examples 1 to 3 were subjected to a fungal resistance test by the following procedure, in accordance with JIS Z 2911:2010 (“Methods of test for plastic product”). However, to propagate fungi for a short period of time and accelerate the test, a 10% glucose-peptone medium was further added in the test.

Each test fungus shown in Table 3 was inoculated into a potato dextrose agar medium and cultured at 25° C. for 7 to 14 days. Then, a 10% glucose-peptone medium was added to control the number of spores to 10⁶ CFU/mL, thereby preparing a spore fluid. In the same manner, the spore fluids of other test fungi shown in Table 3 were prepared.

A surface of the article of Example 1, which was composed of a cured product of the resin composition for forming the projection structure, was sterilized by ethanol and cut into 50 mm-square pieces, thereby producing test samples. The same procedure was carried out on the articles of Examples 2 and 28 and Comparative Examples 1 to 3, thereby obtaining test samples thereof.

Each spore fluid was sprayed entirely on a surface of each test sample to the extent that droplets were formed thereon. The test sample was hung so that the sprayed surface faced in the vertical direction. The fungi were cultured for 4 weeks in the following conditions:
Temperature: 24±1° C.
Humidity: 95% RH

After the culture, the surfaces of the test samples were observed by the unaided eye and a stereoscopic microscope and determined in accordance with the following criteria. The results are shown in Table 3.
0: Fungal growth was not found by the unaided eye and the microscope.
1: Fungal growth was not found by the unaided eye; however, it was clearly found by the microscope.
2: Fungal growth was found by the unaided eye, and the area of the growth site was found is less than 25% of the total area of the sample.
3: Fungal growth was found by the unaided eye, and the area of the growth site was 25% or more and less than 50% of the total area of the sample.
4: Hyphae grew well, and the area of the growth site is found was 50% or more of the total area of the sample.
5: Hyphae grew very well and entirely covered one surface of the sample.

TABLE 3

| | Fungal resistance test | | | | |
|---|---|---|---|---|---|
| | Aspergillus | Cladosporium | Chaetomium | Penicillum | Rhizopus |
| Example 1 | 1 to 2 | 1 | 2 | 2 | 2 |
| Example 6 | 1 | 1 | 2 | 2 | 1 |
| Example 28 | 1 to 2 | 1 to 2 | 1 to 2 | 1 to 2 | 1 to 2 |
| Comparative | 3 | 3 | 3 | 3 | 3 |
| Example 1 | | | | | |
| Comparative | 3 | 3 | 3 | 3 | 3 |
| Example 2 | | | | | |
| Comparative | 4 | 4 | 4 | 4 | 4 |
| Example 3 | | | | | |

### <Antifungal Evaluation 2>

The antifungal evaluation 2 was carried out in the same manner as the antifungal evaluation 1, except that the fungi were changed to Pythium vanterpoolii, Fusarium solani, Fusarium oxysporum, and Fusarium moniliforme. The evaluation results are shown in Table 4.

TABLE 4

| | Fungal resistance test | | | |
|---|---|---|---|---|
| | Pythium | Fusarium | Fusarium | Fusarium |
| | vanterpoolii | solani | oxysporum | moniliforme |
| Example 1 | 2 | 2 | 2 | 2 |
| Example 6 | 3 | 2 | 2 | 2 |
| Example 28 | 2 | 2 | 2 | 2 |
| Comparative | 4 | 4 | 4 | 4 |
| Example 1 | | | | |
| Comparative | 4 | 4 | 4 | 4 |
| Example 2 | | | | |
| Comparative | 5 | 5 | 5 | 5 |
| Example 3 | | | | |

### Conclusion

As a result of the above-mentioned fungal resistance tests in the wet condition at a temperature of 24±1° C. and a humidity of 95% RH, according to the above evaluation criteria, fungal propagation at a level of 3 to 5 was found in the comparative article obtained in Comparative Example 1 (which is such an article that the ratio (Wt/Wb) of the width Wt at the 97% height of the height to the width Wb at the bottom is 0.56), the comparative article obtained in Comparative Example 2 (which is such an article that the ratio (Wt′/Wb′) of the width Wt′ at the 97% height of the height to the width Wb′ at the bottom is 0.55) and the comparative article obtained in Comparative Example 3 (which is such an article that the surface is flat).

Meanwhile, the antibacterial and antifungal articles obtained in Examples 1, 6 and 28 are each an article that has the projection structure comprising such projections that the height H is 80 nm or more and 1000 nm or less, and the ratio (Wt/Wb) of the width Wt at the 97% height of the height to the width Wb at the bottom is 0.5 or less, or an article that has the linear convexo-concave shape comprising such linear convexes that the height H′ is 80 nm or more and 1000 nm or less, and the ratio (Wt′/Wb′) of the width Wt′ at the 97% height of the height to the width Wb′ at the bottom is 0.5 or less. Therefore, as a result of the fungal resistance tests and according to the above evaluation criteria, fungal propagation found in the articles was at a level of 1 or 2 only, and for all the fungi used in the tests, their propagation was reduced.

### Example 29

A resist pattern was formed by laser photolithography on a surface of an aluminum flat plate uniformly plated with chromium. The plating layer was subjected to etching, thereby forming such a convexo-concave shape corresponding to a projection structure, that many concaves (depth 200 nm) are disposed at an average interval of 200 nm. An original plate was uniformly coated with a thin DLC film (20 nm in thickness) in order to ensure the durability of the original plate and removability between the original plate and resin, thereby producing the flat plate-shaped original plate for forming a projection structure.

The original plate for forming the projection structure was formed into the shape of the cap of a single screw extruder by a desired method and installed in the extruder. By use of the extruder and, at 230° C., ZEONOR (product name, polyolefin manufactured by ZEON Corporation) as an extrusion resin, a tube-shaped antibacterial and antifungal article having the projection structure on the inner surface, was obtained. The inner diameter and outer diameter of the article were 0.90 mm and 0.76 mm, respectively. For the projection structure formed on the inner surface of the tube, such projections were 83% of all projections, that the average P_{AVG} of two adjacent projections' distances was 195 nm; the height H was 188 nm; the width Wt at the 97% height of the height was 58 nm; the width Wb at the bottom was 192 nm; and the ratio (Wt/Wb) of Wt to Wb was 0.3.

For ZEONOR (product name, polyolefin manufactured by ZEON Corporation), the results of the combustion tests and the test for extractable substances defined in “Test Methods for Plastic Containers” in the Japanese Pharmacopoeia (14th Edition) satisfy the criteria that are equal to or less than the above-mentioned standard values.

### Example 30

A resist pattern was formed by laser photolithography on a surface of a plate uniformly plated with copper. The plating layer was subject to etching, thereby forming a convexo-concave shape corresponding to a projection structure on the plate. The plate was attached to the surface of a copper wiring (diameter 0.55 mm) to produce a cored bar. The surface of the cored bar was coated with NOVATEC-HD (product name, a high density polyethylene resin produced by Japan Polypropylene Corporation) by a wire coating (extrusion coating) method to have an outer diameter of 0.7 mm. Then, with fixing one end of the cored bar, the other end was pulled to decrease the diameter of the cored bar, and the cored bar was pulled out, thereby obtaining a tube-shaped antibacterial and antifungal article having the projection structure on the inner surface. For the projection structure formed on the inner surface of the tube, such projections were 78% of all projections, that the average P_{AVG} of the two adjacent projections' distances was 190 nm; the height H was 175 nm; the width Wt at the 97% height of the height was 63 nm; the width Wb at the bottom was 180 nm; and the ratio (Wt/Wb) of Wt to Wb was 0.35.

For NOVATEC-HD (product name, a high density polyethylene resin manufactured by Japan Polypropylene Corporation), the results of the combustion tests and the test for extractable substances defined in “Test Methods for Plastic Containers” in the Japanese Pharmacopoeia (14th Edition) satisfy the criteria that are equal to or less than the above-mentioned standard values.

Examples 29 and 30 were subjected to the following antibacterial evaluation 2.

### <Antibacterial Evaluation 2>

Test bacterial solutions were produced in the same manner as the antibacterial evaluation 1.

The antibacterial and antifungal articles obtained in Examples 29 and 30 were cut into 1 cm-square pieces, wiped with ethanol for disinfection and used as test samples. A sterile PET film (product name: A4100, manufactured by: Toyobo Co., Ltd.) was cut into a 1 cm-square piece and used as a control.

With reference to ASTM E2149 and the shake method defined by the Society of International sustaining growth for Antimicrobial Articles, the articles were evaluated by a shake flask method. In particular, 25 fragments (width 1 cm) of the tube were put in 50 ml of each test bacterial solution in a conical flask and subjected to shake culture at 150 rpm and 35° C. for 24 hours.

Measurement of the numbers of viable bacteria and calculation of the antibacterial activity values were carried out in the same manner as the antibacterial evaluation 1. The antibacterial activity values of Example 29 were as follows: Escherichia coli 4.8 and Staphylococcus aureus 5.2. The antibacterial activity values of Example 30 were as follows: Escherichia coli 4.2, Staphylococcus aureus 4.8.

### REFERENCE SIGNS LIST

1. Substrate
2. Projection structure
2′. Linear convexo-concave shape
3. Projection
3′. Linear convex
10, 10′. Antibacterial article
31. Local maximum point
32. Line segment
40. Plastic greenhouse
41. Ceiling
42. Wall surface
43. Soil surface (reflective sheet)
50. Plant cultivation unit
51. Reflective sheet
52. Light source
60. Container
61. Packaging material
62. Outlet port
70. Packaging material
80. Carport
81. Roofing material
90. Tube
91. Support
92. Projection structure or linear convexo-concave shape
100. Medical patch
101. Slit
102. Catheter insertion site

1. An antibacterial and antifungal article comprising a projection structure on a surface of the antibacterial and antifungal article, the projection structure comprising a projection group comprising a plurality of projections being disposed, where an average P_{AVG} of distances P between adjacent projections is 1 μm or less,
wherein the projection structure comprises projections that a height H is 80 nm or more and 1000 nm or less and a ratio (Wt/Wb) of a width Wt at a 97% height of the height to a width Wb at a bottom, is 0.5 or less.

2. An antibacterial and antifungal article comprising a linear convexo-concave shape on a surface of the antibacterial and antifungal article, the linear convexo-concave shape comprising a plurality of linear convexes extending in one direction or approximately one direction, where an average P′_{AVG} of distances P′ between adjacent linear convexes is 1 μm or less,
wherein the linear convexo-concave shape comprises linear convexes that a height H′ is 80 nm or more and 1000 nm or less and a ratio (Wt′/Wb′) of a width Wt′ at a 97% height of the height to a width Wb′ at a bottom, is 0.5 or less.

3. An antibacterial and antifungal agricultural material, wherein at least a part of the antibacterial and antifungal agricultural material comprises the antibacterial and antifungal article defined by claim 1.

4. An antibacterial and antifungal medical device, wherein at least a part of the antibacterial and antifungal medical device comprises the antibacterial and antifungal article defined by claim 1.

5. The antibacterial and antifungal medical device according to claim 4, wherein the antibacterial and antifungal medical device is in a tube shape and has the projection structure on at least a part of an inner surface of the tube.

6. An antibacterial and antifungal agricultural material, wherein at least a part of the antibacterial and antifungal agricultural material comprises the antibacterial and antifungal article defined by claim 2.

7. An antibacterial and antifungal medical device, wherein at least a part of the antibacterial and antifungal medical device comprises the antibacterial and antifungal article defined by claim 2.

8. The antibacterial and antifungal medical device according to claim 7, wherein the antibacterial and antifungal medical device is in a tube shape and has the linear convexo-concave shape on at least a part of an inner surface of the tube.
